(19) United States
(12) Patent Application Publication
(10) Pub. No.: US 2012/0041225 A1
(43) Pub. Date: Feb. 16, 2012

(54) KIT FOR AUTOMATED RESOLVING AGENT SELECTION AND METHOD THEREOF

(76) Inventor: Niteen A. Vaidya, Cupertino, CA (US)

(21) Appl. No.: 13/278,430

(22) Filed: Oct. 21, 2011

Related U.S. Application Data
(63) Continuation-in-part of application No. 11/347,532, filed on Feb. 3, 2006, now abandoned.

(51) Int. Cl. C07B 57/00; C07C 209/88; C07C 69/003; C07C 67/52; C07C 69/76; C07C 57/30; C07C 211/27

(52) U.S. Cl. 560/78; 562/402; 564/304; 560/191

(57) ABSTRACT

The present invention provides a means for the rapid selection of optimum resolving agents and solvents, combinations and conditions to separate optical isomers. The present invention combinedly describes and automates a full lifecycle of chiral separation method development and optimization through a series of kits and procedures providing screening, automation for screening, racemate recovery, enantiomer preparation, and method optimization.

### CROSS-REFERENCE TO RELATED APPLICATIONS

The present application claims priority to and is a continuation in part of Ser. No. 11/347,532 filed Feb. 3, 2006, the disclosure of which is incorporated by reference in its entirety.

### FIELD OF THE INVENTION

The present invention relates to identification of optimal diastereoisometric salt crystallization conditions. Specifically, the present invention relates to an improved method and kit for the rapid selection of optimum resolving agents and solvents, combinations and conditions to separate optical isomers.

### BACKGROUND

In the pharmaceutical sciences, it has long been known that useful organic compounds are isomeric. Often one isomer is therapeutic, while the other isomer has a neutral health benefit or more likely has significant harmful side effects. This was the case in the 1960s with the drug, thalidamide. One isomer induced the desired sleep, but the other isomer was teratogenic, causing significant defects in the in utero fetus by reducing blood vessel growth. The thalidamide isomer that causes reduction in blood vessel growth is now being examined in cancer therapy to reduce blood vessel growth to tumors.

Similarly, the anti-arthritis drug, naproxen—the active ingredient in the anti-inflammatory ALEVE® (Proctor & Gamble, Cincinnati, Ohio)—is an optically active isomer. One isomer causes liver dysfunction; the other isomer is therapeutic for arthritis. Naproxen has strict controls and limits on the active isomer released for public use.

A major limitation to resolution of mixtures of racematic compounds concerns the identification of optimal diastereoisometric salt crystallization conditions. This screening often takes too long.

During early drug discovery and drug development phase, availability and cost of racemate is a concern and it makes it more difficult for scientists to exhaustively identify the optimum chiral separation process for a given racemate. Thus, in addition to a quicker screening process, there is a need for a process that requires very little racemate.

With the increasingly competitive drug-development landscape and exponential cost related to drug development research, leading pharmaceutical companies prefer to maximize their use of resources by studying library of compounds during lead identification phase. There is therefore a need for an automated system providing screening and identifying comprehensive separation conditions for a library of racemates for target disease area, thereby allowing companies to jump-start their research and save many months of research time.

Method optimization to minimize manufacturing cost and to determine the shortest route to pure enantiomer is difficult and requires testing against different ratios between racemate and resolving agents, and different concentration of solvents or combination of solvents. There is a need for a process that allows parallel testing of all these conditions in a standard and organized manner, and that provides the ability to compare results under constant experimental conditions, so that optimization work can be conducted within matter of days.

Chiral technology has wide application in specialty chemicals such as pharmaceuticals, herbicides, pheromones, liquid crystals, non-linear optical materials and polymers, aroma and flavors, vitamins, sweeteners, dyes and pigments, etc. The worldwide market for chiral products is over $200 billion. In the pharmaceutical area alone it is $115 billion for single enantiomer drugs. There are three basic approaches in getting enantiopure compounds: 1) chiral synthesis, 2) separation of racemic mixtures, and 3) enzymatic degradation of one enantiomer. There are several clear advantages of separation of racemates over that chiral synthesis. First, they tend to be simpler processes. Second, they often give much better volumetric productivities for equivalent optical purities. Third and perhaps most importantly, the desired enantiomeric excess (ee) is achieved by adjusting the level of conversion (in the case of diastereomeric crystallization if one increases the number of recrytallizations the (ee) is subsequently increased at each stage).

Examination of a representative group of such drugs shows that roughly 65% have optical activity after classical resolution (diastereomeric crystallization). There are clearly many instances where resolution is both economically viable and the preferred method. The main challenges involved with this method are to select optimum resolving agent/nature and composition of the solvent within a given time frame (pre-manufacturing decision). This selection is often time consuming, tedious, and labor intensive. Recognizing this as a unique commercial opportunity where today no other comparative product is available in the market, chiral kits (96, 192, 384, etc.) vials for easy robotic manipulation and step-by-step instructions for simple experimental set up are available. A series of tests on racemates to choose the combination of resolving agents and solvents may be done in parallel to maximize the chances of success. The protocol of resolution experiments is developed so that considerable time is saved before the pre-manufacturing decision is made on the choice of resolving agent/solvent and conditions of resolution.

Chiral technology has wide application in specialty chemicals such as pharmaceuticals, herbicides, pheromones, liquid crystals, non-linear optical materials and polymers, aroma and flavors, vitamins, sweeteners, dyes and pigments, etc. The worldwide market for chiral products is over $200 billion. In the pharmaceutical section alone it is $115 billion for single enantiomers drugs (Ref. 1). It has been recognized for a long time that the shape of a molecule has considerable influence on its physiological properties. Differentiation within enantiomer pairs are numerous and often dramatic. Examples such as previously described herein and further provided below emphasize the reasons for commercial interest and incentive for producing optically pure materials by methods applicable to at least multigram amounts and in many cases to hundreds or thousands of tons.

Examples of fine chemicals, which show the effect of chirality are provided in (Table 1)

TABLE 1
CHIRAL EFFECT ON PROPERTIES

| | | |
|---|---|---|
| Compound | Isomer | Effect |

Pharmaceuticals

| | | |
|---|---|---|
| Thalidomide | S-Isomer | Teratogenic |
| | R-Isomer | Sleep inducing |
| Barbiturates | S-Isomer | Depressant |
| | R-Isomer | Convulsant |
| Opiates | R,S-Isomer | Narcotics |
| | S,R-Isomer | Non-addictive cough-mixture |
| Labetalol | R,S-Isomer | Alpha-blocker |
| | S,R-Isomer | Beta-blocker |
| Penicillamine | D-Isomer | Anti-arthritic |
| | L-Isomer | Toxic |

Food/Flavor

| | | |
|---|---|---|
| Aspartame | R,R-Isomer | Sweet taste |
| | S,R-Isomer | Bitter taste |
| Carvone | R-Isomer | Spearmint odor |
| | S-Isomer | Caraway odor |
| Limonene | R-Isomer | Orange odor |

Vitamins

| | | |
|---|---|---|
| Ascorbic acid | L-Isomer | Antiscorbutic acid |

Insecticide

| | | |
|---|---|---|
| Bermethrin | d-Isomer | More toxic than I-Isomer |

Herbicide

| | | |
|---|---|---|
| Fluazifop butyl | R-Isomer | Plant growth regulator |
| Paclobutrazol | R-Isomer | Fungicide |
| | S-Isomer | Plant growth regulator |

There are two basic approaches in obtaining chiral compounds: asymmetric synthesis and resolution.

Asymmetric synthesis often requires auxiliary chiral synthesis or asymmetric catalysis. Resolution involves separation methods such as chromatography, polymer-supported liquid membrane and preferential or diastereometric crystallization. Asymmetric synthesis should be, in principle, the most cost-effective method for producing single-enantiomer products, because all the precursors are converted to the desired enantiomer. However, in industry the decision to implement an asymmetric synthesis approach is typically based on an assessment of efficiency and cost. Among the factors considered are (1) the catalyst efficiency (that is, the number of product molecules produced per molecule of the catalyst); (2) the availability of the metal, the ligand, and the starting materials (especially critical for low value products); and (3) reaction conditions, such as very low temperature or high pressure, and reaction kinetics.

Chiral chromatography is a useful technique for small-scale resolution of racemic mixtures (less than one kilo of material). Several ways to obtain optically pure material, such as asymmetric synthesis (introduce chirality during synthetic sequence), synthesis using chiral pool and stereoselective synthesis using enzymes or chemicals and resolution of racemic mixture using either chromatography or liquid membrane or chemical resolution.

Diastereometric crystallization is widely used in the separation of racemic mixtures even though the theoretical yield is only 50%. But if unwanted isomer is racemized back to the mixture, which sets-up a recycle process to yield the desired optical isomer, which would have an unprecedented economic advantage over other methods².

Utilizing phase diagrams generally speeds up the selection of a good resolving agent and determination of the best crystallization conditions. However, the selection process is still very empirical and trial and error is the best solution to the criteria.

An alternative to diastereomeric salt formation is direct, preferential crystallization of the desired enantiomer, usually initiated by seeding with a pure enantiomer. If applicable, preferential crystallization of enantiomers is a highly economic approach, and Merck, for example, has used it with the great success in the manufacture of alpha-methyl—DOPA (alpha-methyl-L-dihydroxyphenylalanine). However, in practice the method has limited application because it can be applied only to a conglomerate, i.e. a mechanical mixture of crystals of the two enantiomers. In contrast, a true racemic compound where both enantiomers exist in a unit cell cannot be resolved by preferential crystallization. Unfortunately, less than 20% of all known racemates are conglomerates and therefore the remainder are true racemic compounds and cannot be separated by preferential crystallization. Differential scanning calorimeter to obtain a melting point diagram is one method used to assign to which of two classes a racemate belongs.

TABLE 2
OPTICALLY ACTIVE PHARMACEUTICALS
PRODUCED (WHOLLY OR PARTIALLY)
USING CRYSTALLIZATION TECHNIQUE
Worldwide Sales

| | | Resolving | |
|---|---|---|---|
| Product | Therapeutic Class | Agent | ($Millions) |
| Amoxycillin | Antibiotics | | 2000 |
| Ampicillin | Antibiotics | (D-Camphor- | 1800 |
| | | sulphonic acid) | |
| Captopril | Cardiovascular | | 1580 |
| Diltiazem | Calcium antagonist | (+)-Phenethyl- | 980 |
| | | amine | |
| Naproxen | Antiinflammatory | (−) (Cinchoni- | 971 |
| | | dine) | |
| Cefalexin | Antiiotic | | 900 |
| Timolol | Cardiovascular | | 325 |
| Cefadroxil | Antibiotic | | 300 |
| ∞-Methyldopa | Cardiovascular | | 225 |
| Chloroampheicol | Antibiotic | (D-Camphor- | 80 |
| | | sulphonic acid) | |
| Dextromethorphan | Antitussive | | 50 |
| Ethambutol | Tuberculostatic | (L-Tartaric | 50 |
| | | acid) | |

(See Chirotechnology, by R. Sheldon, ed, Marcel Dekker, London, 1992)

Examination of a representative group of such drugs shows that roughly 65% owe there optically activity to classical resolution. There are clearly many instances where resolution is both economically viable and preferred method.

Diastereometric crystallization has the advantage of relative simplicity, robustness and requires only standard production equipment. From the practical point of view, the method is flexible and suited for intermittent batch production, which is often the practice in pharmaceutical manufacture. While the occurrence of desirable crystal behavior and solubilities are in large measure unpredictable, a systematic search for exploitable properties at all relevant points in a sequence will reward the effort and should be part of the modus operandi of the process development chemist. For example, if a substance is readily racemized and a crystallization-induced asymmetric transformation (deracemization) is possible, it offers an extremely attractive industrial option.

There are two types of diastereomers: (1) ionic/salt; and (2) covalent/neutral. Covalent diastereomers are easier to separate by HPLC than are ionic diastereomers. Even so, covalent diastereomers are not preferred because their formation is not as easy as that of salt; nor is their decomposition. Moreover, the forward and reverse reactions are more subject to racemization of chiral centers than is salt formation.

### The Screening of Resolving Agents and Optimization of Resolution

The initial problem associated with diastereomeric crystallization is to choose the right resolving agent and the nature and composition of the solvent. This can be time consuming, tedious, and labor intensive. Points one must take into consideration include:
1. The diastereomeric salt must crystallize well and there must be an appreciable difference in solubility between two salts.
2. The complex between the resolving agent and the substance to be resolved should be easily formed, and the resolving agent should be easily recoverable in a pure state from the salt following the crystallization step.
3. In general, a resolving agent should be available in an optically pure form because a substance to be resolved cannot be obtained in a higher state of optical purity than their resolving agent by mere crystallization of diastereoisomers.
4. The chiral center should be as close as possible to the functional group responsible for salt formation.
5. An agent must be chemically stable and not racemize under the conditions of the resolution process.
6. Resolving agent should be available as both enantiomers so that both forms of the substrate can be prepared.
7. For industrial purposes, a resolving agent should be relatively inexpensive and readily recoverable in high yield after completion of the resolution.

There are no empirical rules that one of skilled in the art can adhere to when it comes to choosing the optimum resolving agent and solvent combination. Fortunately, the number of commercial quantity resolving agents is limited and one can devise standard protocol to screen resolving agents with that of solvents. The table below provides some common resolving agents:

Examples of fine chemicals, which show the effect of chirality (table 1)

TABLE 3
COMMONLY USED RESOLVING AGENTS

| Acids | Bases |
|---|---|
| Tartaric acid (+)(−) | ∞-Methylbenzylamine (+)(−) |
| Dibenzoyltartaric acid (+)(−) | Ephedrine (+)(−) |
| Mandelic acid (+)(−) | 2-Amino-1-butanol (+)(−) |
| Camphoric acid (−) | Quinine (−) |
| Malic acid (+)(−) | Quinidine (+) |
| 1-Camphor-10-Sulphonic acid (+)(−) | Cinchonidine (−) |
| Pyroglutamic acid (+)(−) | Cinchonine (+) |
| ∞-Methoxyphenylacetic acid (+)(−) | Brucine (−) |
| ∞-Methoxy-∞-trifluoromethylphenyl | Dehydroabietylamine (+) |
| Acetic acid (+)(−) | |

In addition, for important commercial applications companies may design their own resolving agents, such as chiral phosphoric acid developed by andeno and citramalic acid by Lonza. Syntex developed n-methyl-D-glucamine (prepared from D-glucose) for resolution of naproxen (NAPROSYN®, a trademark of Syntex, Inc.) (over 1000 tons per year) as a substitute for cinchonidine. Recrystallization of diastereomeric salts usually need polar solvents such as alcohols, acetone with varying degrees of water (Ref. 4).

Some references of interest include:

U. C. Dyer, et al., Org. Proc. Res. Dev. 3(#3), 161-165 (1999). This article discusses the application of automation and thermal analysis for resolving agent selection. However, it does not teach the present invention.

D. R. Aztec, et al., Adv. Synth. Catal. 345(#4), 524-532 (2003). This article discusses automated enzyme screening methods for the preparation of enantiopure pharmaceutical intermediates. It is understood that U.S. Pat. Nos. 6,296,673 and 6,630,006, and others cited for high throughput transfer are modified in the present invention to add racemate to the individual tubes, thus increasing the efficacy of the identification of the condition and process.

B. D. Santarsieno, et al., in U.S. Pat. Nos. 6,296,673, 6,630,006, and related issued patents. These patents teach the use of high throughput automated screening of materials, primarily protein for optimal crystal growth for x-ray diffraction study.

Y-Chem International of Cupertino, Calif. has produced and sold simple non-automated kits for the selection of resolving agents, solvents and conditions. See http//:www.ychem.com.

Chirality in Industry, A. N. Collins, ed. Vol. I. The Commercial Manufacture and Application of Optically Active Compounds, John Wiley & Sons, Inc, New York, 1991. Chirality in Industry, G. N. Sheldrake, ed, vol. II, Developments of the Commercial Manufacture and Application of Optically Active Compounds, John Wiley & Sons, Inc., New York, 1997.

All U.S. Patents are incorporated herein by reference in their entirety.

### SUMMARY OF INVENTION

In addition to providing a means for the rapid selection of optimum resolving agents and solvents, combinations and conditions to separate optical isomers, the present invention combinedly describes and automates a full lifecycle of chiral separation method development and optimization through a series of kits and procedures to deliver a well defined optimum chiral separation method that can be scaled to manufacturing scale. This standardization of work eliminates human errors and allows execution of work with minimum supervision to deliver final results within days.

It is further an object of the present invention to provide kits for improved identification of the optimal conditions for diasteroisomeric salt crystallization and the selection of the optimal solvents and resolving agents, which kit comprises:
A. An array of containers wherein the array is a standard high throughput tray and the containers are a multiplicity of substantially identical containers or well plates each optionally sealed with a sealant or stoppers, to avoid loss of chemicals,
B. wherein each substantially identical container has a unique combination of resolving agent in each column and at least one unique suitable solvent (either already added to container or provided in separate plate to be added later during experiment) in each row;
C. wherein each kit and the substantially identical container is made of material (e.g. polypropylene or glass) that is chemically inert and withstands temperatures ranging from −20° C. to +120° C. to allow the experiment to be done within the container(s);
D. wherein optionally all substantially identical containers within the kit are held together by means of a holder or ring to allow inspection and comparison of results (namely crystals formed) among the containers visually without having to take out individual container out of the kit; and
E. Instructional text to use the kit.

It is a further object of the present invention to provide a method for the rapid high throughput determination of the solvents and conditions for the crystallization of diasteroisomeric salts to separate enantiomers, which method comprises:
A. Obtaining a kit as described herein;
B. Adding to each container of the array:
   i. a measured amount of racemic organic compound neat (or dissolved in solvent and then evaporating that solvent)
   ii. optionally adding the solvents provided in separate plate using same positional matrix
C. Heating the combination of sub-step B to a solubilization temperature not in excess of 100° C. for less than 15 minutes;
F. Optionally, agitating the combination of sub-step C for between about 5 min. and 24 hr.;
G. Cooling the heated combination of sub-step C;
H. Observing the formation of diastereomeric crystals visually or by optical means in each container;
I. Separating the formed diastereoisometric salts;
J. Isolating and evaluating the desired isomer; and
K. Selecting the optimal combination of resolving agent and solvents and resolution conditions based on the experimental results of substeps A to J.

It is a further object of the present invention to define a robotic system set-up and a step-by-step procedure that describes how to use it for selecting optimum resolving agents and solvents, combinations and conditions to separate optical isomers.

It is a further object of the present invention to provide a means for separation and/or recovery of a mixture of optical isomers (or racemate) from a solution that contains combination of various resolving agents, solvents and enantiomeric salts of optical isomers.

It is a further object of the present invention to provide a procedure for quick incremental purification of enantiomeric salts of selected optical isomer(s)

It is a further object of the present invention to provide one or more customized trays and a step-by-step procedure that can be used to define and optimize a chiral separation method using diastereomeric crystallization technique.

It is a further object of the present invention to provide a quick screening process requiring a small amount of racemate (0.001 to 0.1 mmol per container as described above), and allow exhaustive and parallel screening by offering over 100, preferably over 200, and preferably over 350 separation conditions per racemate.

There has thus been outlined, rather broadly, exemplary features of the invention in order that the detailed description thereof that follows may be better understood, and in order that the present contribution to the art may be better appreciated. There are, of course, additional features of the invention that will be described further hereinafter.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

As such, those skilled in the art will appreciate that the conception upon which this disclosure is based may readily be utilized as a basis for the designing of other structures, methods and systems for carrying out the several purposes of the present invention. It is important, therefore, that equivalent constructions insofar as they do not depart from the spirit and scope of the present invention, are included in the present invention.

For a better understanding of the invention, its operating advantages and the specific objects attained by its uses, reference should be held to the accompanying drawings and descriptive matter which illustrate preferred embodiments of the invention.

### BRIEF DESCRIPTION OF THE FIGURES

FIG. 1A is a schematic representation of a flow chart for the separation (resolution) of racemic bases. For neutral racemates such as aldehydes, alcohols, ketones and the like, the same steps are used after the pre-processing steps described herein below in the identified section are performed.

FIG. 1B is a schematic representation of a flow chart for the resolution (separation) of racemic acids. For amino acids, the same steps are used after the pre-processing steps described herein below in the identified section are performed.

FIG. 2A is a photographic view of the top of a kit showing the columns and rows of tubes in the tray with a cover sheet.

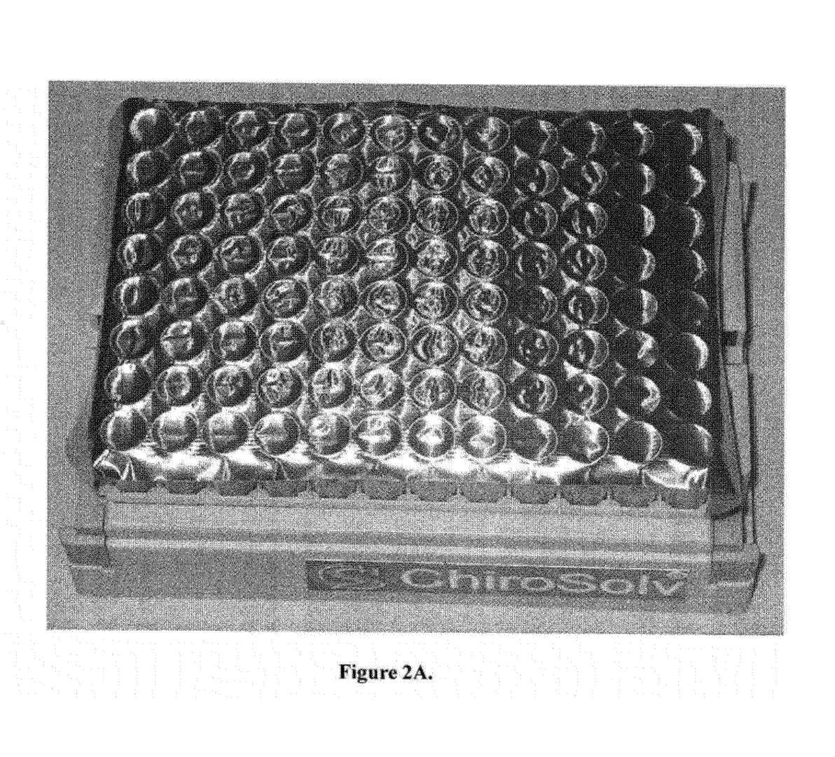

FIG. 2B is a photographic top view of the kit with tubes individually sealed with septum.

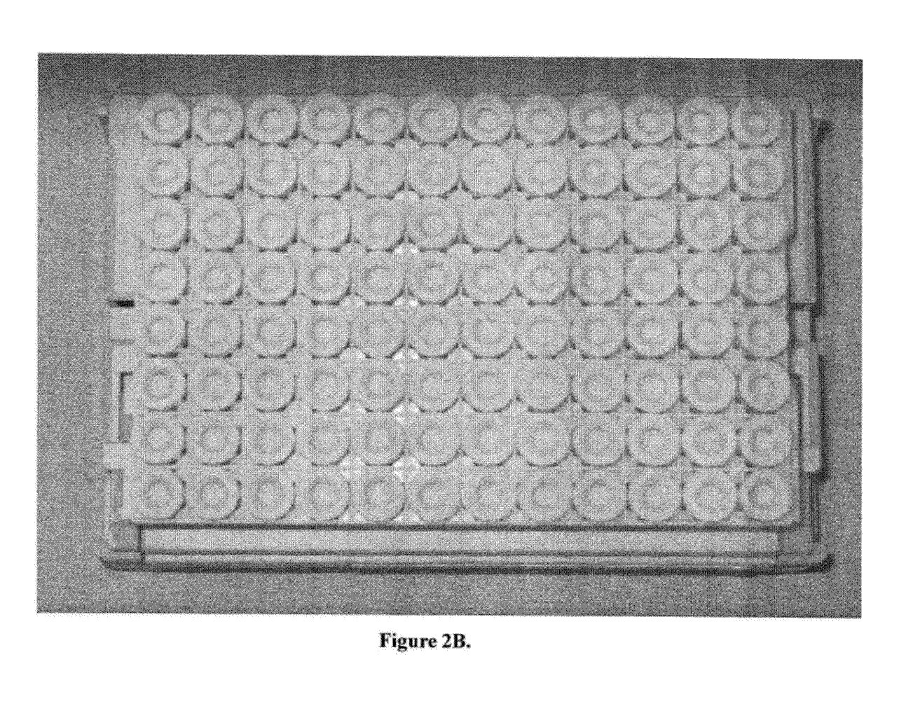

FIG. 3A is a photographic view of a side view of the tubes sealed with a sheet and tray of the kit.

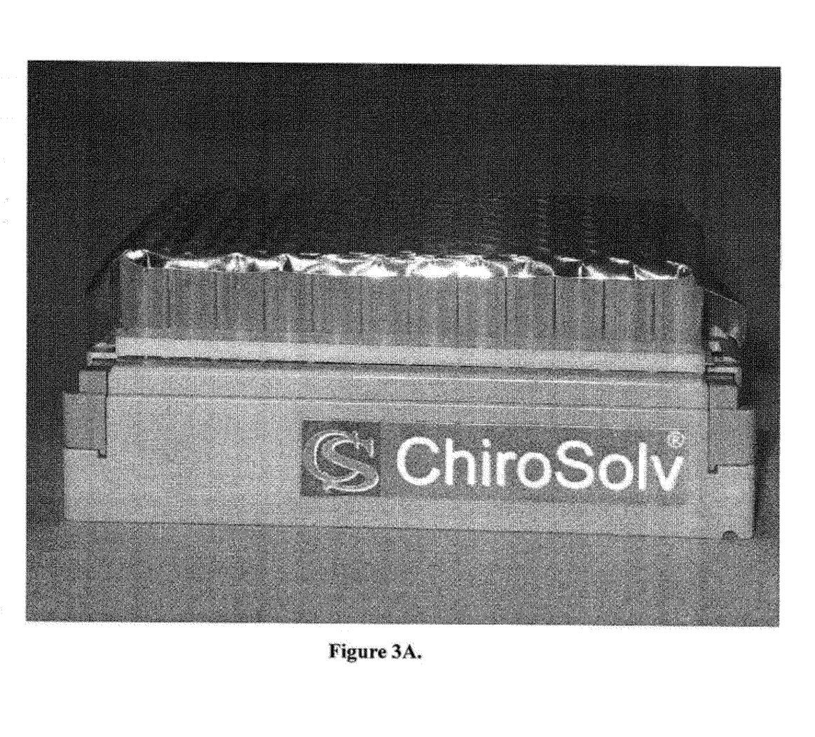

FIG. 3B is a photographic side view of the tubes individually sealed with septums in the tray.

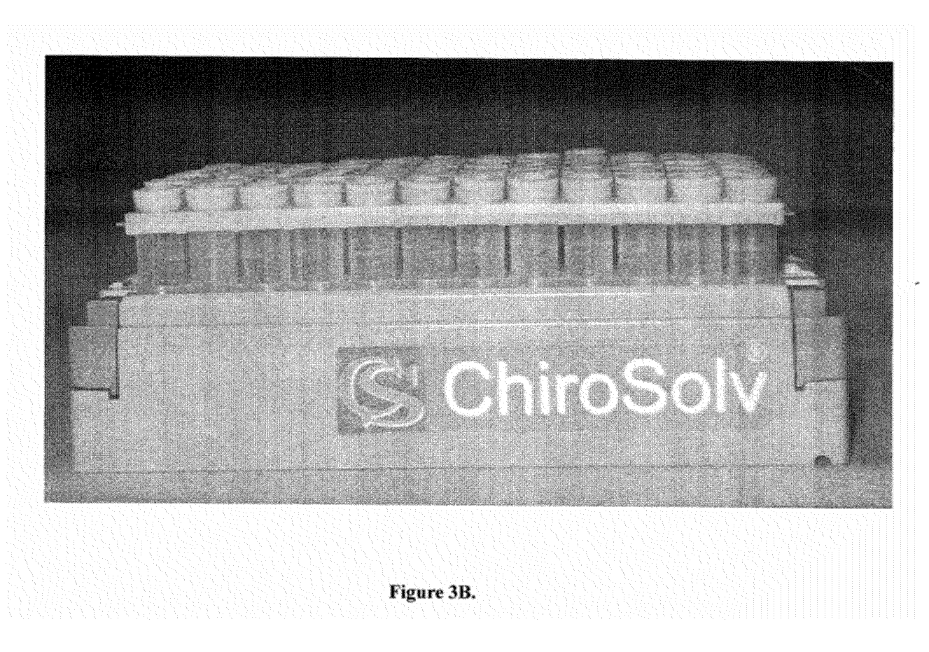

FIG. 4 is a photographic view of one tube having a bar code on the bottom and a second tube having an alphanumeric code.

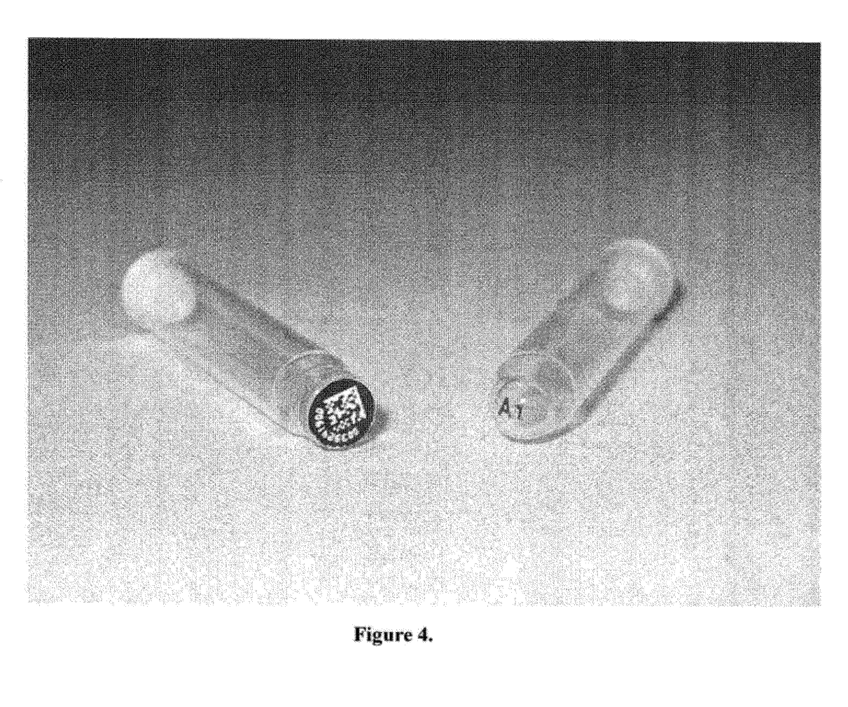

FIG. 5 is an isometric view of the tube in the tray with a bar code.

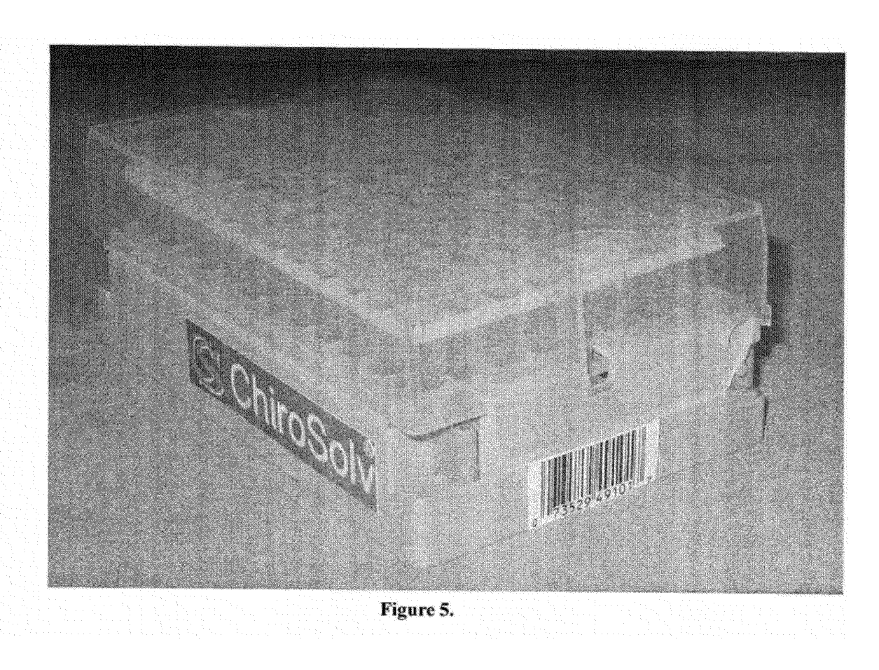

FIG. 6 is an isometric photographic view of 96 tubes with a representative bar code on their bottom (on the left) and 96 tubes having a representative alphanumeric code on their bottom (on the right).

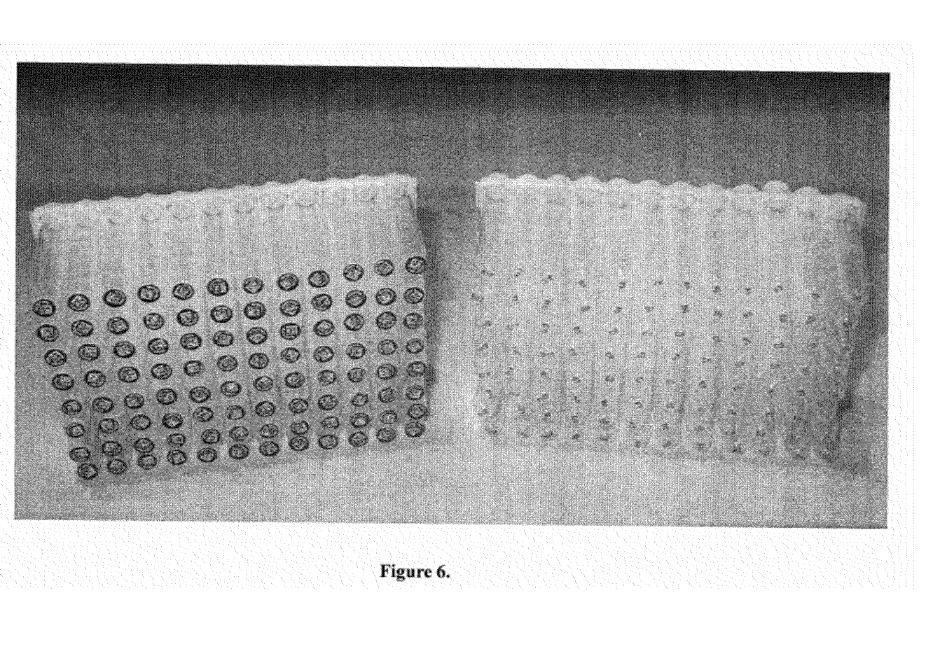

FIG. 7 is an illustration of a method of enantiomer preparation involving screening, racemate recovery, and enantiomer development through repeated re-crystallization.

FIG. 8 is an illustration of method optimization including screening, enantiomer development (for each selected separation condition), and testing against optimize kits to identify optimal conditions.

FIG. 9 illustrate kits for acid or base racemate, recovery solution, filter-funnel with PTFE membrance and vacuum adapter, recover reservoir, purification reservoir, tips and pH paper.

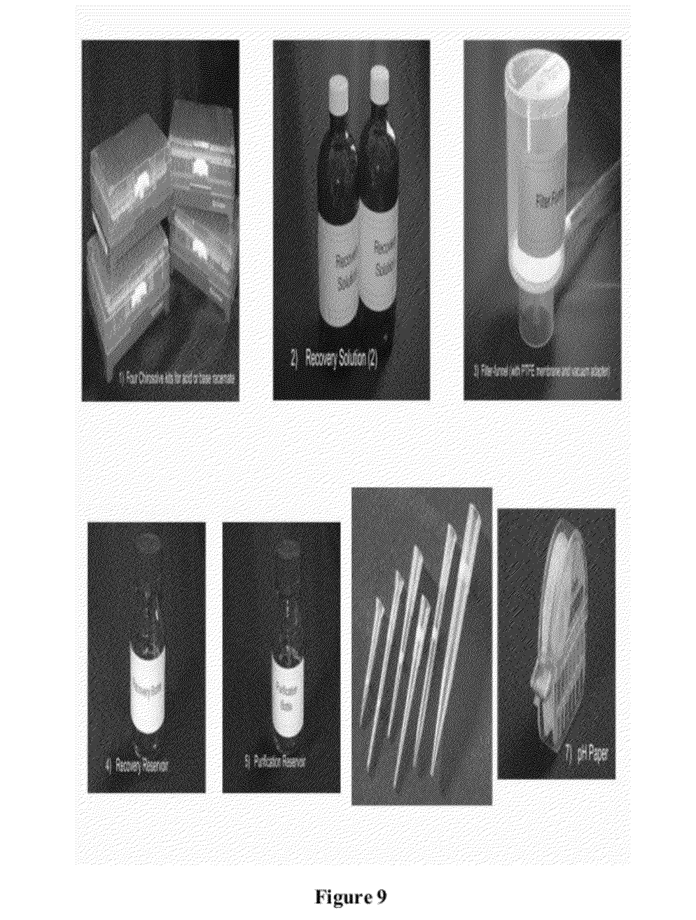

FIG. 10 illustrates an enantiomer preparation kit.

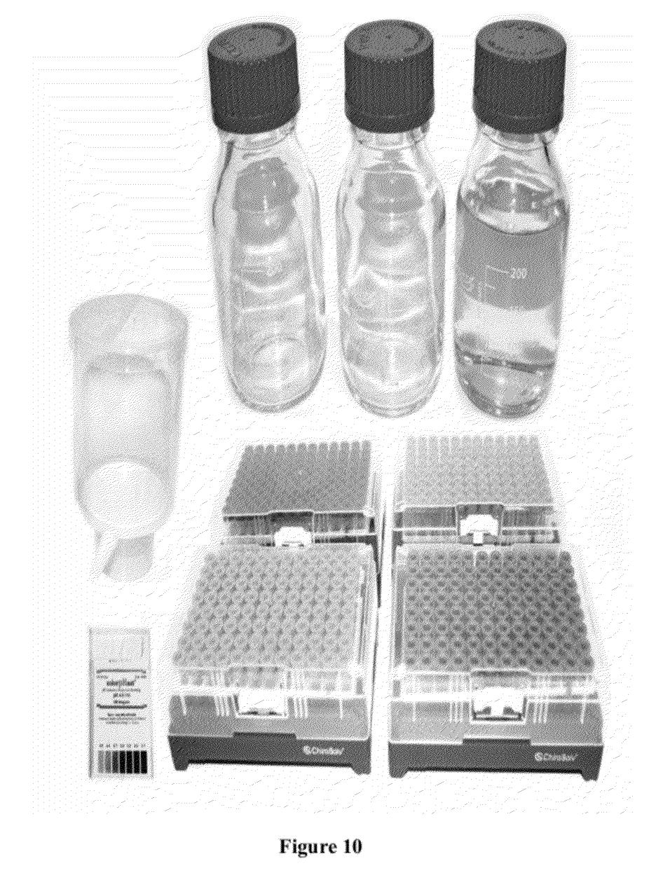

### DETAILED DESCRIPTION OF THE INVENTION

### Definitions

As used herein:

“Acid resolving agent” or “acidic resolving agent” refers to commonly known acid resolving agents of the art. The resolving agent is selected from the group consisting of tartaric acid, pyroglutamic acid, di-p-tolulo-tartaric acid, mandelic acid, malic acid, camphorsulphonic acid, dibenzoyl-tartaric acid, deoxycholic acid (+), camphoric acid (+), quinic acid (−), aspartic acid (+), glutamic acid, 1,3,4,6-diisopropylidine-2-ketogluconic acid (−), acetylmandelic acid, N-acetyl-1-hydroxyproline, N-acetyl-1-leucine, acetyl-3-mercapto-2-methylpropionic acid, 3-acetylmercapto-2-methylpropionyl-1-proline, N-acetyl-D-3-(2-naphthyl)-alanine, (R)-acetylthio-2-methylpropionyl chloride, N-acetyl-1-phenylalanine, N-acetyl-1-tyrosinamide, D-alanine, 1-aminoadipic acid, (R)-2-aminobutyric acid, (1R,4S)-4-aminocyclopent-2-ene-1-carboxylic acid, (1S,4R)-4-aminocyclopent-2-ene-1-carboxylic acid, S-2-amino-3,3-dimethylbutyric acid, 1-tert-leucine, 1,2-amino-2-methyl-3-(3′,4′-dimethoxyphenyl)-propionitrile HCl, 1-2-amino-2-methyl-3-(3′,4′-dihydroxyphenyl)-propionic acid, (R)-2-amino-4-phenylbutane, D-arginine, D-aspartic acid, D-2-azidophenylacetic acid, D-2-azidophenylacetyl chloride, (1S,2R)-cis-2-benzamido-cyclohexane-carboxylic acid, (1R,2S)-cis-2-benzamido-cyclohexane-carboxylic acid, benzyl-(R) & (S)-mandelate, benzyl-2-tosyloxypropionate, N-2-BOC-D-alanine, N-2-BOC-1-aminoadipic acid, 3-(R)-BOC-aminocyclopent-4-ene-1-(S)-carboxylic acid, 3-(S)-BOC-aminocyclopent-4-ene-1-(R)-carboxylic acid, N-2-BOC-D-arginine hydrochloride, N-2-BOC-D-aspartic acid, N-2-BOC-3-(4-biphenyl)alanine, N-2-BOC-N-6-CBZ-D-lysine, N-2-BOC-3-(4-chlorophenyl)-alanine, N-2-BOC-cyclohexylalanine, N-2-BOC-1-cyclohexylalanine methyl ester, N-2-BOC-3,3-diphenylalanine, N-2-BOC-3-(4-fluorophenyl)-alanine, N-2-BOC-D-glutamic acid 1-benzyl ester, N-2-BOC-D-histidine, N-2-BOC-3-(4-iodophenyl)-alanine, N-3-BOC-D-leucine, N-3-BOC-1-tert-leucine DCHA salt, (1S)-camphanic acid, (1R)-camphorsulfonic acid, (1S)-camphorsulfonic acid, 2-methylbenzylamine, N-2-BOC-D-methionine, N-2-BOC-3-(1′-naphtyl)alanine, N-2-BOC-3-(2′-naphtyl)alanine, N-2-BOC-3-(4′-nitrophenyl)alanine, N-2-BOC-1-octahydroindole-2-carboxylic acid, N-2-BOC-3-(pentafluorophenyl)-alanine, N-2-BOC-D-phenylalanine, N-BOC-D-proline, N-1-BOC-D-3-(2′-pyridyl)alanine, N-2-BOC-1-3-(2′-pyridyl)alanine, N-2-BOC-D-3-(3-pyridyl)alanine, N-1-BOC-1-3-(3′-pyridyl)alanine, N-2-BOC-D-serine, N-BOC-1,2,3,4-tetrahydroisoquinoline-(3R)-carboxylic acid, N-BOC-1,2,3,4-tetrahydroisoquinoline-(3S)-carboxylic acid, N-2-BOC-3-(4′-thiazolyl)alanine, N-BOC-D-threonine, N-2-BOC-N-8-tosyl-D-arginine, N-2-BOC-D-tryptophan, N-2-BOC-D-tyrosine, N-2-BOC-D-tyrosine methyl ester, N-2-BOC-D-valine, 2-bromobutyric acid, 2-bromohexadecanoic acid, (R)-2-bromo-2-phenylacetic acid, 2-bromopropionic acid, butyl-(S)-2-chloropropionate, (2R,3S)-butyl-2,3-epoxybutyrate, (R)-butyl-2,3-epoxybutyrate, (S)-tert-butyl-3-hydroxybutyrate, (S)-butyl-lactate, N-butyl-(R)-2-methyl-2-hydrazino-3-(3′-methoxy-4′-hydroxyphenyl)-propionate, N-CBZ-D-alanine, N-CBZ-D-arginine, N-CBZ-D-aspartic acid, N-CBZ-O-tert-butyl-D-serine, CBZ-1-cyclohexylalanine, N-CBZ-D-glutamic acid, N-CBZ-D-histidine, N-CBZ-D-leucine, N-CBZ-1-tert-leucine DCHA salt, N-CBZ-D-methionine, N-2-CBZ-D-3-(2′-naphthyl)alanine, N-2-CBZ-ornithine, N-2-CBZ-D-phenylalanine, N-2-CBZ-D-proline, N-2-CBZ-D-serine, N-2-CBZ-D-threonine, N-2-CBZ-D-tryptophan, N-2-CBZ-D-tyrosine, N-2-CBZ-D-valine, (R)-2-chlorobutyric acid, 3-chloromandelic acid, 4-chloromandelic acid, 1-((S)-3-chloro-2-methylpropionyl)-1-proline, (R)-2-(4′-chlorophenoxy)-propionic acid, 3-(4′-chlorophenyl)alanine, 2-(4′-chlorophenyl)-3-phenylpropionic acid, chlorophos, 2-chloropropionic acid, (S)-2-chloropropionic acid sodium salt (50% solution), cyclohexylalanine, cyclohexylglycine, cyclophos, D-cysteine, D-cysteine hydrochloride monohydrate, D-cysteine, dibenzoyl-tartaric acid, 1-3-(3′,4′-dichlorophenyl)-alanine, diethyl-1-tartrate, D-1-dihydrophenylglycine, D-1-dihydrophenylglycine chloride hydrochloride, D-(3′,4′-dihydroxy)-1-phenylglycine, diisopropyl-tartrate, dimethyl-tartrate, 2-3-diphenylpropionic acid, di-p-toluoyl-tartaric acid, ethyl-(R)-2-(N-acetylamino)-2,4-dimethylpentanoate, ethyl-(R)-2-(N-acetylamino)-2-methyl-3-phenylpropionate, ethyl-4-bromo-3-hydroxybutyrate, ethyl-4-chloro-3-hydroxybutyrate, ethyl-(S)-2-chloropropionate, ethyl-2-3-dihydroxybutyrate, ethyl-2-3-dihydroxy-3-phenylpropionate, (R)-ethyl-3-hydroxybutyrate, ethyl-2-hydroxy-2-phenylacetate, ethyl-(R)-2-hydroxy-4-phenybutyrate, ethyl-3-hydroxy-3-phenylpropionate, (R)-ethyl-4-iodo-3-hydroxybutyrate, N-(1-phenylethyl)-phtalimide, D-phenylglycine, N,N,N′,N′-tetramethyl-tartaric acid, thiazolidine-4-carboxylic acid, 3-(2-thienyl)-alanine, D-allo-threonine, valine and combinations thereof.

“Base resolving agent” or “basic resolving agent” refers to commonly known base resolving agents of the art. These resolving agents include, but are not limited to N-methylglucamine (−), α-methylbenzylamine, cinochonidine (−), ephedrine (−), hydroquinidine (+), N-benzyl-α-methylbenzylamine, brucine (−), strychnine (−), pseudoephedrine (+), qunidine, quinine (−), cinchonine (+), threo 2-amino-1-(p-nitrophenyl)-1,3-propanediol, 2-amino-1-butanol, methylephedrine (−), α-1-naphthylethyl amine, dehydroabietyl amine, 2-amino-1-phenyl-1,3-propanediol, D-alaninamide, 2-amino-1-propanol, 2-aminobutanol, erythro-2-amino-1,2-diphenylethanol, (S)-1-aminoindane, cis-(1S,2R)aminoindan-2-ol, 1-amino-2-(methoxymethyl)-pyrrolidine, 2-amino-3-methyl-1-butanol, 2-amino-3-methyl-1-pentanol-isoleucinol, 2-amino-4-methyl-1-pentanol-leucinol, 2-amino-1-[4′-(methylthio)-phenyl]-1,3-propanediol, 2-amino-1-phenylethanol, 1-amino-2-propanol, 1-aminotetralin and N-propyl derivative, 2-aminotetralin and N-propyl derivative, N-benzyl-3-aminopyrrolidine, benzyl-benzyl amine, benzyl-4-chlorobenzylamine, cis-N-benzyl-2-(hydroxymethyl)cyclohexylamine, N-benzyl-3-hydroxypyrrolidine, N-benzyl-2-methylbenzylamine, N-benzylamine-methylbenzylamine hydrochloride, 2-benzyl-2-methylbenzylamine, 2-benzyl-3′-methylbenzylamine, 2-benzyl-4′-methylbenzylamine, N-benzyl-1-(1′-naphthyl)ethylamine hydrochloride, bis(methoxymethyl)pyrrolidine, Bis {1-[1-naphthyl]ethyl}amine hydrochloride, bis(1-phenylethyl)amine hydrochloride, N,N-bis-[1-phenylethyl]phthalamic acid, N-2-BOC-cyclohexylglycine, BOC-isoleucinol, BOC-phenylalaminol, BOC-prolinol, N-butyl-2-amino-2-methyl-3-(3′,4′-dihydroxyphenyl)-propionate, CBZ-1-cyclohexylalaminol, N-2-CBZ-D-3-(1′naphthyl)alaminol, N-2-CBZ-D-3-(2′naphthyl)alaminol, N-1-phenylalaminol, 2-(2′-chlorobenzyl)benzyl-amine, 2-(3′-chlorobenzyl)benzyl-amine, 2-(4′-chlorobenzyl)benzylamine, (S)-cyclohexylalaminol, 1,2-diaminocyclohexane, (S)-2,6-diamino-1-hexanol (1-lysinol), 1,2-diaminopropane, 2,2-dibenzyl-2-hydroxy-1-methylethylamine, N,N-dibenzylphenylalaminol, N-(3,4-dimethoxybenzyl)-1-phenylethylamine, 3,3-dimethyl-2-aminobutane, N,N-dimethyl-1-methylbenzylamine, N, N-dimethyl-2-(1′-naphthyl)ethylamine, N-(3′,4′-dinitrobenzoyl)-2-methylbenzylamine, N-(3′,5′-dibenzoyl)-1-(1-naphthyl)ethylamine, 1,2-diphenyl-1,2-ethanediamine, 2,2-diphenyl-2-hydroxy-1-methylethylamine, N,N′-ditosyl-1,2-diphenyl-1,2-ethanediamine, 2,2-diphenyl-2-hydroxy-1-methylethylamine, N,N′-ditosyl-1,2′-diphenyl-1,2-ethanediamine, diphenylvalinol, diphenylprolinol, ethyl-(R)-2-amino-2-methyl-3(3′,4′-dimethoxyphenyl)propionate, ethyl (R)-2-amino-2-methyl-3-phenylpropionate, 3-hydroxypyrrolidine, 3-hydroxypyrrolidine HCl, isopropyl-2-methylbenzylamine, 1-tert-leucinol, 1-tert-leucinol hydrochloride, 1-methioniol, 5-methoxy-2-aminotetralin, N-propyl-5-methoxy-2-aminotetralin, 6-methoxy-2-aminotetralin and N-propyl-6-methoxy-2-aminotetralin, 7-methoxy-2-aminotetralin and N-propyl, 8-methoxy-2-aminotetralin and N-propyl derivative, (S)-2-(methoxymethyl)pyrrolidine, (S)-2-(methylamino)propiophenone, D-N-methylamphetamine, 2-(4′-methylbenzyl)benzylamine, 2-(4′methylbenzyl)-N′N′-dimethylbenzylamine, 2-(4′-methylbenzyl)-N-hydroxyethyl-benzylamine, 2-methyl-3′-bromobenzylamine, 2-methyl-4′-bromobenzylamine, 2-methyl-4′-bromobenzylamine hydrochloride, 2-methyl-4′-chlorobenzylamine, 2-methyl-2′-methoxybenzylamine, 2-methyl-3′-methoxybenzylamine, 1-methyl-3′-methoxybenzylamine, 2-methyl-4′-methoxybenzylamine, 2-methyl-4′-methylbenzylamine, N-methyl-2-methylbenzylamine, N-methyl-2-(1′-naphthyl)-ethylamine, 2-methyl-2′-nitrobenzylamine hydrochloride, 2-methyl-4′-nitrobenzylamine hydrochloride, 1-methyl-3-phenylpropylamine, 2-(1′-naphthyl)ethylamine, 2,(2′-naphthyl)ethylamine, phenylalaminol, (R)-1-phenyl-3-aminobutane, 2-phenylglycinol, 1-phenylpropylamine, 2-phenyl-1-propylamine, (S)-prolinol, 1-threoninol, N-acetyl-2-phenylglycinol, dinaphthylprolinol, 2-methylpiperazine, piperidinol, quinuclidinol and combinations thereof.

“Solvent” refers to those organic liquids (optionally in any combination with water) which solubalize the components. Solvents include, but are not limited to 90% acetone, methyl ethyl ketone (2-butanone), 1-butanol, 2-propanol, 90% 2-propanol, methanol, 80% methanol, ethanol, 96% ethanol, water, 1-propanol, 85% 1-propanol, acetonitrile, ethyl acetate, dichloromethane, chloroform, p-dioxane, methyl-t-butyl ether, toluene, tetrahydrofuran. The kit may also utilize one or more solvents selected from the group consisting of 1-butanol, 2-butanol, n-butyl acetate, carbon tetrachloride, chlorobenzene, chloroform, cyclohexane, cyclopentane, o-, m-, p-dichlorobenzene, dimethyl acetamide, dimethyl sulfoxide, dioxane, 2-ethoxyethanol, ethylene dichloride, glyme, heptane, hexadecane, hexane, iso-hexanes, 2-methoxyethanol, methyl t-butyl ether, methyl isoamyl ketone, methyl n-propyl ketone, dichloromethane, N-methylpyrrolidine, nonane, pentane, petroleum ether, propylene carbonate, pyridine, tetrahydrofuran, toluene, benzene, trichloroethylene, 1,1,2-trichlorotrifluoroethane, 2,2,4-trimethylpentane, o-xylene, actal, acetamide, acetophenone, acetylacetone, adiponitrile, allyl acetate, allyl alcohol, anisole, benzenethiol, benzonitrile, benzyl acetate, benzyl alcohol, benzyl benzoate, benzyl chloride, benzyl ethyl ether, bis(2-chloroethyl)ether, bis (2-ethylhexyl acetate), bromobenzene, 1-bromobutane, 2-bromobutane, 1-bromo-2-chloroethane, bromochloromethane, 1-bromodecane, 2-bromo-2-methylproprane, 1-bromonaphthalene, 1-bromopentane, 1-bromopropane, 2-bromopropane, 1,3-butanediol, 1,4-butanediol, 2,3-butanediol, butanenitrile, butanethiol, cis & trans 2-butene-1,4-diol, butyl acetate, sec-butyl acetate, tert-butyl benzene, butyl ethyl ether, butyl formate, butyl methyl ketone, butyl stearate, p-tert-butyltoluene, butyl vinyl ether, γ-butyrolactone, 1-chloro-3-methylbutane, 3-(chloromethyl)heptane, 1-chloronaphthalene, 1-chlorooctane, 1-chloropentane, o-, m-, p-chlorotoluene, cineole, o-, m-, p-cresol, cis, trans-crotonyl alcohol, cumene, cyclohexaol, cyclohexanone, cyclohexene, cyclohexylbenzene, cyclopentanone, p-cymene, cis,trans-decahydronaphthalene, decane, 1-decene, diacetone alcohol, dibenzyl ether, 1,2-dibromo-1,1-difluoroetane, 1,2-dibromoethane, dibromofluoromethane, dibromomethane, 1,2-dibromopropane, dibutyl ether, dibutyl maleate, dibutyl phthalate, dibutyl sebacate, dibutyl sulfide, 1,2-dichloropropane, 2,4-dichlorotoluene, 3,4-dichlorotoluene, diethyl carbonate, diethylene glycol, diethylene glycol dibutyl ether, diethylene glycol, diethylene glycol diethyl ether, diethylene glycol dimethyl ether, dimethylene glycol monoethyl ether, diethylene glycol monoethyl ether acetate, diethylene glycol monomethyl ether, diethyl ketone, diethyl malonate, diethyl oxalate, 2,3-diethylpentane, diethylpentane, diethyl sulfide, diiodomethane, diisobutyl ketone, dipentyl ether, diisopropyl ether, diisoprpyl ketone, dimethyl adipate, dimethyl aniline, 2,2-dimethylbutane, 2,3-dimethylbutane, 3,3-dimethyl-1-butanol, 2,3-dimethyl-2-butanol, 3,3-dimethyl-2-butanol, cis, trans-1,2-dimethylcyclohexane, dimethyl disulfide, N,N-dimethylformamide, dimethyl glutarate, 2,2-dimethylheptane, 2,2-dimethylhexane, 2,3-dimethylhexane, 2,4-dimethylhexane, 2,5-dimethylhexane, 3,3-dimethylhexane, 3,4-dimethylhexane, dimethyl maleate, 1,2-dimethylnaphthalene, 1,6-dimethylnaphthalene, 2,2-dimethylpentane, 2,3-dimethylpentane, 2,4-dimethylpentane, 3,3-dimethylpentane, dimethyl phthalate, 2,2-dimethyl-1-propanol, dimethyl succinate, 1,3-dioxolane, dipentene, dipentyl ether, diphenyl ether, dipropyl ether, dodecane, 1-dodecene, 1,2-epoxybutane, ethyl acetoacetate, ethyl acrylate, ethylbenzene, ethyl benzoate, ethyl butanoate, 2-ethyl-1-butanol, ethylbutyl ketone, ethyl trans-cinnamate, ethyl cyanoacetate, ethylcyclohexane, ethylene carbonate, ethylene glycol, ethylene glycol diacetate, ethylene glycol dibutyl ether, ethylene glycol diethyl ether, ethylene glycol dimethyl ether, ethylene glycol ethylether acetate, ethylene glycol monomethyl ether acetate, ethylene glycol monobutyl ether, ethylene glycol monoethylether, ethylene glycol monomethyl ether, 3-ethylhexane, 2-ethyl-1,3-hexanediol, 2-ethyl-1-hexanol, 2-ethylhexyl acetate, ethyl isovalerate, ethyl lactate, 3-ethyl-2-methylpentane, 3-ethyl-3-methylpentane, 3-ethylpentane, ethyl propanoate, fluorobenzene, o-, m-, p-fluoro-toluene, formamide, furfuryl alcohol, glycerol, heptane, 1-heptanol, 2-heptanol, 3-heptanol, 1-heptene, cis, trans2-heptene, hexafluorobenzene, hexamethylphosphoric trimide, hexane, hexanenitrile, 1,2,6-hexanetriol, 1-hexanol, 2-hexanol, 3-hexanol, 1-hexene, cis,trans-2-hexene, cis,trans-3-hexene, hexyl acetate, sec-hexyl acetate, hexylene glycol, hexyl methyl ketone, hydraacrylonitrile, iodobenzene, 1-iodobutane, 2-iodobutane, iodoethane, 1-iodo-2-methylpropane, 1-iodopropane, 2-iodopropane, isobutyl acetate, isobutylbenzene, isobutyl formate, isobutyl isobutanoate, isopentyl acetate, isopentyl isopentanoate, isophorone, isopropyl acetate, D & L-limonene, 2,4-lutidine, 2,6-lutidine, mesitylene, mesityl oxide, n-methylacetamide, methyl acetate, methyl acetoacetate, methyl benzoate, 2-methyl-1-butanol, 3-methyl-1-butanol, 2-methyl-2-butanol, 3-methyl-2-butanol, methyl cyanoactate, methylcyclohexane, 1-methylcyclohexanol, cis, trans-2-methylcyclohexanol, cis,trans-3-methylcyclohexanol, cis, trans-4-methylcyclohexanol, methylcyclopentane, N-methylformamide, 2-methylheptane, 3-methylheptane, 4-methylheptane, 2-methylhexane, 3-methylhexane, methyl isobutyl ketone, methyl isopentyl ketone, 1-methylnaphthalene, 2-methyloctane, 3-methyloctane, 4-methyloctane, methyl oleate, 2-methylpentane, 3-methylpentane, 2-methyl-1-pentanol, 3-methyl-1-pentanol, 2-methyl-2-pentanol, 3-methyl-2-pentanol, 4-methyl-2-pentanol, 2-methyl-3-pentanol, 3-methyl-3-pentanol, 4-methyl-4-penten-2-one, methyl pentyl ketone, N-methylpropanamide, 2-methylpropanenitrile, 2-methyl-1-propanol, 2-methyl-2-propanol, methyl propyl ketone, N-methyl-2-pyrrolidone, methyl salicylate, 2-methyl tetrahydrofuran, 2-methylthiophene, 3-methylthiophene, 4-methylvaleronitrile, β-myracene, nitroethane, nitromethane, 1-nitropropane, 2-nitropropane, nonane, 1-nonene, octane, octanenitrile, 1-octanol, 2-octanol, 1-octene, cis, trans-2-octene, pentachloroethane, 1,5-pentanediol, pentanenitrile, 1-pentanol, 2-pentanol, 3-pentanol, pentyl acetate, β-phellandrene, phenetole, 2-picoline, 3-picoline, 4-picoline, α-pinene, β-pinene, 1,2-propanediol, 1,3-propanediol, propanenitrile, propargyl acetate, propargyl alcohol, propyl acetate, propylbenzene, propyl benzoate, propylene carbonate, propyl formate, pseudocumene, styrene, α-terpinene, terpinolene, 1,1,2,2-tetrabromoethane, 1,1,1,2-tetrachloroethane, 1,1,2,2-tetrachloroethane, tetrachloroethylene, tetrachloromethane, tetraethylene glycol, tetraethylsilane, tetrahydrofuran, tetrahydrofurfuryl alcohol, tetrahydronaphthalene, tetrahydropyran, tetrahydrothiophene, 2,2,3,3-tetramethylpentane, 2,2,3,4-tetramethylpentane, 2,2,4,4-tetramethylpentane, 2,3,3,4-tetramethylpentane, tetramethylurea, thiodiethanol, thiophene, toluene, o-, m-, p-toluidine, α-tolylnitrile, triacetin, tribromomethane, tributyl borate, tributyl phosphate, 1,1,1-trichloroethane, 1,1,2-trichloroethane, trichloroethylene, trichloroethylsilane, trichlorofluoromethane, (trichloromethyl)benzene, trichloromethylsilane, 1,2,3-trichloropropane, 1,1,2-trichlorotrifluoroethane, tri-o-cresyl phosphate, tridecane, 1-tridecene, triethylene glycol, triethyl phosphate, 2,2,2-trifluoroethanol, (trifluoromethyl)benzene, 1,2,3-trimethylbenzene, 2,2,3-trimethylbutane, 2,2,5-trimethylhexane, 2,3,5-trimethylhexane, 2,2,3-trimethylpentane, 2,2,4-trimethylpentane, 2,3,3-trimethylpentane, 2,3,4-trimethylpentane, trimethyl phosphate, 1-undecene, veratrole, vinyl acetate o-, m-, p-xylene and combinations thereof.

“Recovery solution for acidic diastereomeric salt” refers to a mixture of one or more of following: Citric acid, hydrochloric acid, sulfuric acid, Dichloromethane, chloroform, carbon tetrachloride, ethylene dichloride, ethylene dibromide, ethyl acetate, methyl acetate, diethyl ether, methyl t-butyl ether, toluene, benzene

“Recovery solution for basic diastereomeric salt” refers to mixture of one or more of following: sodium hydroxide, Sodium Carbonate, Sodium Bicarbonate, Dichloromethane, carbon tetra chloride, chloroform, ethylene dichloride, ethylene dibromide, ethyl acetate, methyl acetate, toluene, benzene, diethyl ether, methyl t-butyl ether

Screen Solution: The present invention permits one of skill in the art to quickly screen resolving agents and solvents to find the most optimum combination and to optimize reaction conditions in order to separate a racemic mixture (acids, bases, alcohols, amino acids, aldehydes/ketones) into its constituent enantiomers. It does this by offering eight types of kits, each with, for example, 12 rows of 8 vials (a total of 96 vials). Each vial contains a pre-measured quantity of a unique combination of resolving agent and solvent (solvents provided within the vials or in a separate 96-well plate using same positional matrix). As a result, scientists can potentially screen up to 768 combinations of the resolving agents and solvents, if all eight types of kits are used at the same time.

Resolving agents are chosen with manufacturing use in mind. They are relatively inexpensive and readily recoverable in high yield after completion of the resolution. In industrial practice, the quantity of resolving agent is often less than the stoichiometric amount, which allows for better separation of the desired enantiomer at a lower cost.

By providing pre-measured quantities of resolving agents and solvents, the present invention offers:
a) The ability to do research in parallel, reducing the research time by up to 90%, i.e., experiments can be finished in days rather than months.
b) Technology know-how; resolving agents and the solvents and their proportions are selected with full understanding of the solubility diagram and through years of experience in developing chirally pure compounds by the chief technologist.
c) Consistent research environment and accurate results.
d) Resolving agents are chosen with manufacturing in mind; which are relatively inexpensive and readily recoverable in high yield after completion of the resolution.
e) Optimized use of the skilled staff time by avoiding mundane mechanical work of measuring.
f) Elimination of human errors. The kits are designed to be used with auto-station; each vial and kit has identification barcode for easier tracking.

By optionally providing solvents in a separate plate, the present invention allows the scientists to easily dispense very small quantity of solid racemate dissolved in a suitable solvent into each vial, evaporating out that solvent from each vial before adding the experimental solvents provided in separate plate. This ensures that only one solvent per vial is used during the experiment

Each disposable kit is equipped with plastic or glass vials (e.g. 96) that bear unique alphanumeric and/or bar-code markings, are optionally held together by means of a ring or holder, and are held in a rack designed for robotic manipulation. Vials are usually about 0.75 to 4 ml, preferably about 1.4 ml in size, thus requiring very small amount of the unknown sample. Both vials as well as the rack are heat and chemical resistant and withstand temperatures of −20° to +120° C. This unique design allows the scientists to perform the entire experiment and inspect the results visually without having to take the vials out of the rack.

The advantages of the present invention include:
a) eight types of screening kits providing 768 combinations (or more kits possible) of solvents and resolving agents;
b) ready-to-use disposable kits (all you need is your unknown compound);
c) very little amount of unknown needed (less than 3 mmol per kit);
d) vials and the racks both have the same material specificity, making them heat and chemical resistant, and able to withstand temperatures of −20° to +120° C. (the entire experiment can be done within the rack);
e) easy to follow step-by-step instructions and results charts;
f) conveniently designed for easy robotic manipulations to eliminate human errors;
g) kits and/or vials have unique identification (e.g., barcode or alphanumeric code) for cross-referencing; and
h) long shelf life for the kits.

These kits are based on a straightforward acid-base neutralization technique, followed by re-crystallization in suitable solvent. The goal is to determine the most optimum combination of resolving agent and solvent that allows quick crystallization of the chirally pure compound and to stipulate the conditions permitting maximal recovery of the pure enantiomer. The kits are primarily of two types:
Acid kits (e.g., A1, A2, A3, A4, etc.): Include a group of chirally pure acids. They are used to resolve racemic bases. Each kit includes 8 types of acids and twelve types of solvents. Most of the acids used in these kits are easily available in bulk quantities and are commonly used in manufacturing processes.
Base kits (e.g., B1, B2, B3, B4, etc.): Include a group of chirally pure bases (or amines). They are used to resolve racemic acids. Each kit includes 8 types of amines and twelve types of solvents. Most of the bases used in these kits are easily available in bulk quantities and are commonly used in manufacturing processes.

Each experiment needs about 0.001 mmol to 0.1 millimol, preferably about 0.03 mmol of unknown racemate in each of the 96 vials. The mixture is then heated close to the boiling point of the solvent and then allowed to cool at ambient temperature. It is then further cooled to 4° C. and finally to 0° C. All vials with crystals are indicators of success; while the rest of the vials need to be examined for quick excess solvent test (the lack of crystals may be due to excess quantity of solvent). Typically, only one or two vials will show maximum optical purity. One would be dextro (+) and other laevo (−) rotatory to sodium light. From this matrix, usually only one vial with desired enantiomer will have to be investigated further in addition to its mother liquor for scale-up condition optimization.

The present invention therefore relates to one or more packaged solutions that offer inter-related and complementary solutions that individually and in combination assist in identifying the separation conditions; identifying the fastest route to enantiomer development and in optimizing separation process that is cost effective during large-scale production. The packaged solutions include: Screen, Automation for Screen, Racemate Recovery, Enantiomer Preparation, and Method Optimization.

### Screen Solution

The Screen Solution provides the means for a user to identify a comprehensive list of chiral separation conditions for a given racemate (for both +ve and −ve enantiomer) and to choose the best resolving agent or solvent or the combination thereof that offer optimum results in terms of amount of yield and enantiomeric purity during chiral separation.

The Screen Solution involves a set of one or more kits for improved identification of the optimal conditions for diasteroisomeric salt crystallization and the selection of the optimal solvents and resolving agents. The kit includes an array of containers. The array is a standard high throughput tray and the containers are a multiplicity of substantially identical vials or well plates each optionally sealed with a sealant or stoppers, to avoid loss of chemicals. Each container is made of material (e.g. glass, polypropylene, etc.) that is chemically inert, and that can withstand extreme temperatures. In one embodiment the container can withstand temperatures from 0° C. to 100° C. Each container offers transparency so that the user can visually see the resultant enantiomeric salt crystals and optionally offers inspection through optical means. Each container has a unique combination of resolving agent in each column and at least one unique suitable solvent in each row. The resolving agents can be a set of enantiomerically pure acids that can resolve basic racemate and that have empirical data to back their usage; a set of enantiomerically pure bases that can resolve acidic racemate and that have historically offered high chiral resolution and that have empirical data to back their usage; and/or a set of enantiomerically pure strong acids that can resolve weakly basic racemate that have empirical data to back their usage. Each Screen Solution kit further includes an instructional text to use the kit. The amount of resolving agent in each container ranges from 0.001 mmol to 0.1 mmol. The amount of solvent needed during experiment ranges from 100 μl to 800 μl.

The method for the rapid high throughput determination of conditions for the crystallization of diasteroisomeric salts to separate enantiomers, includes: adding to each container of the array of the Screen Solution kit described above a measured amount of racemic organic compound neat; heating the combination to a solubilization temperature not in excess of 100° C. for less than 15 minutes; optionally, agitating the combination for between about 5 minutes and 24 hours; cooling the heated combination; observing the formation of diastereomeric crystals visually or by optical means in each container; separating the formed diastereoisometric salts; isolating and evaluating the desired isomer; and selecting the optimal combination of resolving agent and solvents and resolution conditions based on the experimental results.

### Automation for Screen

With the increasingly competitive drug-development landscape and exponential cost related to drug development research, leading pharmaceutical companies prefer to maximize their use of resources by studying library of compounds during lead identification phase. Being able to fully automate the above described “Screen Solution” and identifying comprehensive separation conditions for a library of racemates allows companies to jump-start their research and save many months of research time. The automation system set-up and a step-by-step procedure can be used to fully automate the screening work. It is therefore a further object of the present invention to define an automated and/or robotic system for selecting optimum resolving agents and solvents, combinations and conditions to separate optical isomers. As disclosed herein the present invention provides a suggested automation set-up that allows full automation using minimal racemate. During early discovery phase of the drug development lifecycle, making the racemate itself can be difficult and expensive. As a result, very little racemate can be made available for the chiral separation process. The reduced volume kits described herein require only 0.001 mmol of racemate per vial or 0.5 mmol of racemate (about 0.15 gm) per plate, 30 times less volume than standard screen kits. In at least one embodiment the vials of the present invention are glass, have flat bottom and are compatible with any HPLC/HPLC system.

To automate the screen work, a user first selects the appropriate Screen Solution kit. The following automation components are used within the automation system: a multi-channel robotic liquid dispenser that allows the use of filter tips; one filter-tip set being used per racemate, one or more chemically inert empty reservoirs compatible with the liquid dispenser; ability to move a plate to new location; heating station/cooling station with stirring capability; vacuum station; Chiral HPLC equipment and column(s); with capacity to automatically feed vials for analysis. Each of these components are known by one of skill in the art. Velocity-11 by Agilent Inc.) is an example of a multi-channel robotic liquid dispenser that allows the use of filter tips. Agilent Inc. also offers filter-tip sets where the filter tip fits into the liquid dispenser. Velocity-11 by Agilent provides the ability to move a plate to new location.

The following is an exemplary work flow for screen work using above automation platform:
1. Attach filter-tips to dispenser
2. Using the dispenser, add racemate and then solvent to each container of the Screen Solution kit(s)
3. Transfer the kit to heating/cooling station and heat the plate 60 to 80° C., stirring constantly until homogeneous solution
4. Optionally, set aside the plate and corresponding filter-tips and repeat steps 1 to 3 for the next racemate
5. After the diastereomeric crystals are formed in the above treated plates (typically over night), load the corresponding filter tips and the plate now with diastereomeric crystals on to liquid dispenser
6. Aspire filtrate out of vials
7. Dispense filtrate into empty reservoir
8. Wash the filter tips with suitable solvent (methanol or dichloromethane)
9. Add suitable “buffer solution” (<=0.1 mmol) to each container of the kit using the liquid dispenser
10. Move plate with crystals to HPLC station and analyze the contents of each container
11. Chose the best three chiral separation conditions based on the HPLC analysis
12. Optionally repeat the steps 5 to 11 for rest of the racemates chosen in step 4

As used above, “Buffer solution” means commercially available ionic salts needed to maintain the PH balance of the reaction mixture during HPLC analysis. There are buffers of type acid and base that are used for acid and base samples respectively.

### Racemate Separation/Recovery

It is a further object of the present invention to provide a means for separation and/or recovery of a mixture of optical isomers (or racemate) from a solution that contains combination of various resolving agents, solvents and enantiomeric salts of optical isomers. The present invention permits one of skill in the art to quickly recover up to 98% of racemate after screening the racemate to find optimum separation condition through the use of the Screen Solution described herein. This ability maximizes the use of available racemate without compromising the need for comprehensive screening.

During drug-discovery or early drug development phase, scientists have access to very small quantities of racemate. In most cases the racemic mixture itself is very costly and requires elaborate preparation. Therefore being able to recover the racemate that was used during “Screening” as described above; and then purifying the recovered racemate to deliver pure enantiomer is extremely useful and offers great ROI. This solution provides a recovery solution that can be mixed with the mixture of racemate, one or more resolving agents and one or more solvents. Using a simple step-by-step procedure, scientist can recover up to 98% of the racemate used during screening.

Racemate recovery requires one or more bottles containing pre-calibrated amount recovery solution, filter funnel with PTFE membrane filter, bottle to collect recovered racemate and a bottle to collect the filtrate mixture

The method for racemate recovery is as follows:
1. After the screening experiment is finished, heat the kits(s) used during the screen experiment to up to 80° C. (up to five minutes) until most containers have homogeneous solution
2. Pour the contents of all the containers of the kit(s) into empty bottle provided labeled “Filtrate Collection Bottle”
3. Add the content of “Recovery Solution” bottle (making sure that the final solution is strongly acidic (pH less than 4) if the racemate is acidic; or strongly base (pH more than 9) if the racemate is base) to the mixture, mix well and transfer the whole content into a separatory funnel.
4. Let the content in separatory funnel stand until two liquid layers form. The top layer will contain the resolving agents, water and organic solvents; while the bottom layer will contain the racemate and organic solvents. Note that the solvents heavier than water (such as dichloromethane) will stay in the bottom layer; while the solvent lighter than water (such as ethyl acetate) will stay in the top layer.
5. Extract the bottom layer of the solution into an empty bottle labeled “Enantiomer Collection Bottle”
6. Install the filter funnel (provided) with vacuum adapter on top of the “Enantiomer Collection Bottle” and using light vacuum (5 to 15 PSI) evaporate out the solvent. Collect and weigh the racemate recovered

### Enantiomer Preparation/Purification

It is a further object of the present invention to provide a procedure for quick incremental purification of enantiomeric salts of selected optical isomer(s). roducing racemate is often a difficult, time consuming and expensive process which puts a premium on enantiomer separation yield and purity. In addition to the need for chiral separation processes to be very efficient, the process must be fast to allow for continued compound testing. The present invention permits one of skill in the art to quickly isolate enantiomer at target purity during early research. It cost-effectively delivers pure enantiomer from racemate for lead optimization and preclinical testing. The self-contained design of the product allows streamlined and consistent process for method development and to obtain pure enantiomer. Benefits include:
fast delivery of pure enantiomer at a target purity and yield needing minimum amount of racemate;
cost-efficient method development through iterative crystallization accelerates lead optimization and preclinical processes;
consistent results provide solid basis for developing complete separation strategy;
works against any racemate (acid, base, alcohol, aldehyde, ketone, amino acid, racemate with multiple chiral centers);
very little amount of racemate is needed (minimum 0.5 grams); and
results within days; up to 90% of theoretical enantiomer yield.

After identifying the ideal separation condition(s), this packaged solution offers the components that can be used to incrementally purify the enantiomeric salt until target or highest purity is achieved. The solution includes components as well as a step-by-step repetitive procedure that describes how to use the components provided to perform one or more re-crystallization steps, while collecting the enantiomer and filtrate (containing the other isomer) in separate containers. It then proceeds to describe how the other isomer can be recovered and purified to obtain pure isomer. In short, this solution offers both +ve and −ve enantiomers of target (or highest possible) purity within matter of days.

Enantiomer preparation involves a filter funnel with PTFE membrane filter, a set of disposable PTFE filters, bottle to collect filtrate and a bottle to collect purified enantiomer. The method is as follows:
1. Using the above “Screen” solution or by other means, identify the ideal separation conditions that offer highest amount of target enantiomer. The separation conditions must identify the ideal resolving agent and/or solvent or the combination of the two
2. Based on the amount of enantiomer required, use about three times the amount of racemate and less than or equal to that amount of the selected resolving agent (chosen using above described “Screen” solution or by other means) to the empty bottle labeled “Enantiomer collection Bottle”.
3. Add the solvent selected during screening into the “Enantiomer Collection Bottle”. Preferably about 1 ml solvent per mmol of the racemate used.
4. Heat the mixture in “Enantiomer Collection Bottle” in a water bath to up to 80° C. stirring constantly until a homogenous mixture is formed. No solid particles should be visible. In alternative embodiments a magnetic stirrer is used In further embodiments additional solvent is added as needed to obtain desired result.
5. Cool the mixture in “Enantiomer Collection Bottle” at room temperature, allowing time for crystals of enantiomeric salt to form, typically overnight. Depending on the enantiomer property, crystal formation may take longer than overnight. In alternative embodiments cooling the collection bottle will maximize crystal formation, such as cooling to 0° C. In further alternative embodiments crystal initiation (slight shaking or scratching at corners, etc.). assists in crystal formation.
6. Attach the filter funnel on top of the empty bottle labeled “Filtrate Collection Bottle” and attach the funnel to the vacuum source through its vacuum adapter. Insert one of the disposable filter paper into the funnel.
7. Pour the mixture with crystals from the “Enantiomer Collection Bottle” into the filter funnel (with supplied disposable filter) and filter out the filtrate into the “Filtrate Collection Bottle” using low vacuum (5 to 10 psi). Save the filtrate for further recovery of other isomer
8. Using small amount of crystals of the enantiomeric salt collected on top of the filter, do HPLC analysis to check how much enrichment has been achieved.
9. Evaporate residual solvent from the “Enantiomer Collection Bottle”.
10. In a separate flask, heat the selected solvent (chosen during screening), close to boiling point
11. Attach the filter funnel on top of the “Enantiomer Collection Bottle” and pour the solvent into the filter funnel and mix well.
12. Using low vacuum pressure (5 to 10 psi), collect the liquid containing the target enantiomeric salt in to the “Enantiomer Collection Bottle”. Mix well.
13. Set aside the “Enantiomer Collection Bottle” until incrementally enriched diastereomeric salt of target enantiomer crystallizes out (typically over night)
14. Repeat steps 7-13 until desired enantiomeric enrichment is achieved.
15. If the enantiomerically enriched diastereomeric salt is acid, treat it with citric acid or hydrochloric acid (pH 1-3) and the selected solvent to liberate the enantiomer from the salt and collect the final enantiomeric crystals. If the enantiomerically enriched diastereomeric salt is base, treat it with the mixture of one or more of sodium (or potassium) carbonate, bicarbonate or hydroxide (pH 9-11) and the selected solvent to liberate the enantiomer from the salt and collect the final enantiomeric crystals
16. Optionally, to obtain the other isomer, use the second bottle (provided) of above described “Recovery solution” to recover the racemate and follow above described steps 1 to 15.

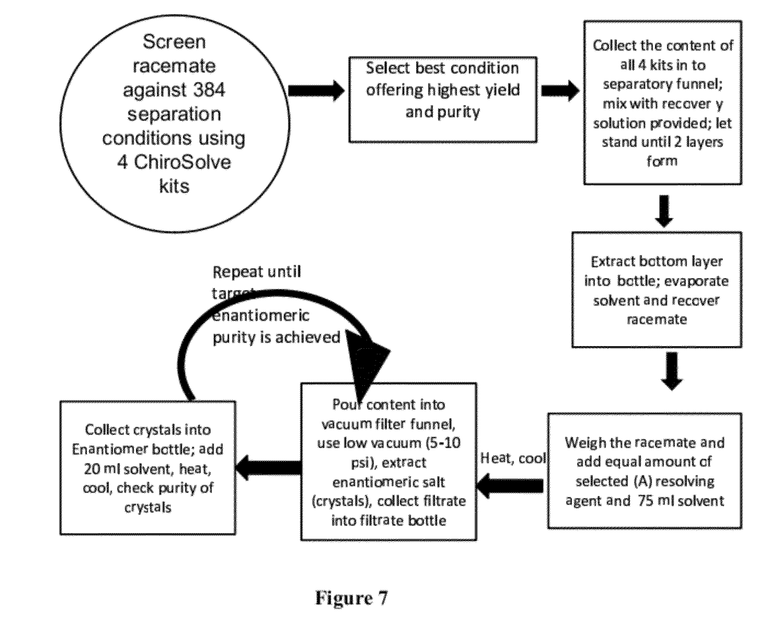

FIG. 7 further illustrates a method of enantiomer preparation that involves Screening, racemate recovery, and enantiomer development through repeated re-crystallization.

### Method Optimization—Chiral Separation Method Using Diastereomeric Crystallization Technique

It is a further object of the present invention to provide one or more customized trays and a step-by-step procedure that can be used to define and optimize a chiral separation method using diastereomeric crystallization technique. The solution disclosed herein is used to develop an optimized robust chiral separation method for target enantiomer that can be scaled up for manufacturing to multi-kilo quantities. It is particularly useful to process chemists who are tasked to find the least expensive, robust and energy-efficient method to manufacture the product. Precision of the methods used for process development are critical as time and material required to attain target purity and yield are magnified as the production requirements increase. Small improvements in the process can have disproportionately positive impacts on costs. The present invention provides a stepwise solution to achieve optimum separation performance. Once a user has identified several candidate separation approaches using the above described “Screen Solution”, the procedure of the present invention is used to determine a preferred approach. Optimization factors include:
ideal ratio between racemate and resolving agent;
ideal concentration of solvent (or combination of solvents); and
minimum material waste and highest energy efficiency.

Benefits of this process include:
chiral separation method optimization achieved within days rather than months by systematically converging on optimization parameters;
focused efforts addressing scalability dynamics in terms of robustness, number of steps involved, energy efficiency and process safety;
establish an elaborate step-by-step scalable procedure for separation through iterative crystallizations required to achieve target purity;
provide a design basis for enantiomer manufacturing; and
works against any racemate (acid, base, alcohol, aldehyde, ketone, amino acid, racemate with multiple chiral centers).

Using the methods and kits of the present invention together provides a standardized comprehensive solution for chiral separation for any type of racemate. For example: 1/if a small quantity of pure enantiomer is desired during early discovery phase and the user has less than 1 gram of racemate available, a preferred path could be Screen solution with reduced volume glass kits+Racemate Recovery+Enantiomer Preparation; 2/if the user is in the process of preparing a filing patent disclosure and requires a comprehensive list of enantiomer development routes, a preferred path could be Screen solution+Enantiomer Preparation; 3/if the user is interested in defining the most optimized chiral separation process that can be scaled up, a preferred path could be Screen solution+Enantiomer Preparation for the best three conditions during Screen+Method Optimization using the finally selected separation condition.

After studying the target enantiomer(s) in small quantity, when larger quantities of enantiomer(s) are needed during pre-clinical/clinical studies or during manufacturing, an optimized method that offers shortest path to pure enantiomer and that offers highest yield needs to be developed. This method optimization study requires extensive experimentation, cost analysis and well thought-out methodic plan. This solution provides a standardized way to do the optimization study that defines the ideal concentration of solvent (or combination of solvents) and the ideal ratio between racemate and the chosen resolving agent. As a result, it offers a highly optimized enantiomer preparation method that minimizes the use of VOC (volatile organic compound) and that can be scaled up to manufacturing capacity.

The method optimization solution involves one or more customized kits for determination of the ideal ratio between racemate and the selected resolving agent that offer optimal conditions for diastereomeric salt crystallization in terms of amount of yield, minimum steps required to get pure enantiomer and maximizing energy efficiency in terms of time and amount of heating/cooling required. Each kit represents the optimum separation condition selected (during screening or during enantiomer preparation) and contains the selected resolving agent and solvent pair. The kit(s) include an array of containers. The array is a standard high throughput tray and the containers are a multiplicity of substantially identical containers or well plates each optionally sealed with a sealant or stoppers, to avoid loss of chemicals. Each substantially identical container contains selected resolving agent in different concentration in each column (so that ratio between racemate and resolving agent is e.g. 1:1.2, 1:1, 1:2, 1:3, 1:4, etc.) and corresponding selected solvent (or solvents in different concentration (e.g. 100%, 90%, 80% 75% 70% etc.) in each row. The kit further includes instructional text as well as a set of one or more pipette tips with filter near the tip.

The method for method optimization is as follows:
1. Using the above “Screen” solution or by other means, identify and select one or more ideal separation conditions that offer highest amount of target enantiomer. The separation conditions must identify the ideal resolving agent and/or solvent or the combination of the two
2. Choose the best three separation conditions and using above described “Enantiomer Preparation Solution”, (one package for each chosen separation condition), define the number of re-crystallizations needed to get target purity of the selected enantiomer. Select the best separation condition that offers shortest path to pure enantiomer
3. Use the optimization kit(s) that contain the resolving agent/solvent or combination thereof corresponding to the selected separation condition. Each row of the kit contains different amount of resolving agent so that when the racemate is added, the ratio between racemate and the resolving agent is different (e.g. 1:1.2, 1:1, 1:2, 1:2.5, 1:3, etc.). Each column of the kit contains the selected solvent(s) or a combination of solvents in different concentration (e.g. 100% 90%, 85%, 80%, etc.). Final constitution of the kits and how many kits are needed is dependent on the target enantiomer
4. Add 0.03 to 0.09 mmol of racemate (as prescribed by instruction sheet in each container of the kit.
5. Heat the kit(s) containing a combination of racemate, resolving agent and solvent(s) to up to 80° C. or until the content of the containers have homogenized. Note at what temperature the solution became homogenized.
6. Cool the kit(s) and observe the crystal formation. Note the containers with highest amount of crystals and how long it took to form the crystals (check every one hour).
7. Select the containers with highest amount of crystals and where the crystal formation was quick and analyze the crystals.
8. Bench-top scale-up is used to confirm that the number of re-crystallization steps established during enantiomer preparation and the optimization factors are consistent during scale-up operation.

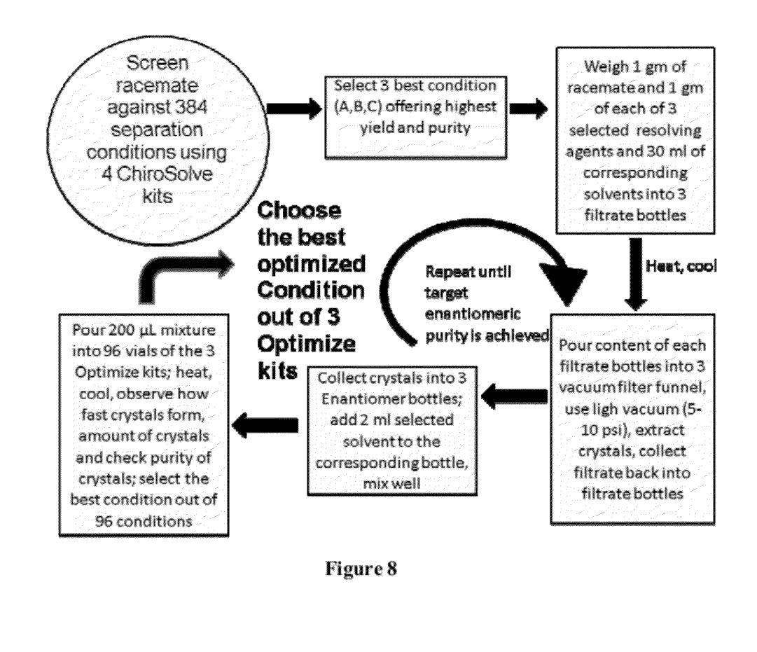

FIG. 8 further illustrates Method Optimization including Screening, enantiomer development (for each selected separation condition), and testing against optimize kits to identify optimal conditions.

### Screening Experiments

The screening experiments involve the following steps:
1. Choose the correct type of kit (A1, A2, A3, A4 etc. or B1, B2, B3, B4 etc.) depending on whether the unknown racemate is base or acid respectively.
2. If the racemate is a type of alcohol, amino acid, aldehyde or ketone, then the pre-processing as is described below is needed.
3. Add 0.001 to 0.05 mmol of the racemate to each of 96 vials. Depending on the availability of the dispensing autostation and the racemate type (liquid or powder), one may need to remove the vial caps. Note that the caps are pre-slitted to accommodate direct injection of racemate by a conventional autostation of the art.
4. Heat the rack and its vials to about 80° C. (the optimum temperature for most of these experiments) or until the mixture becomes homogeneous (up to 100° C.).
5. Allow the kit to cool normally to ambient temperature. Next, if required, further cool the kit to about 4° C. and finally to about 0° C. and observe any crystallization. Vials with crystals formed are considered to be positive tests and need further investigation.
6. Using crystal initiation techniques, attempt to obtain more vials with crystals scratching, seed crystal of enantiomer, etc. Vials with no crystals even after this further action are considered negative tests.
7. Separate the vials with crystals (positive tests) and note their barcode, alphanumeric code, or combination thereof for identification.
8. Analyze each of the crystals separately after liberating enantiomers from the respective diastereomeric salts for specific rotation measurement to sodium or mercury light.

### Pre-Processing for Alcohols

An alcohol is neutral in functionality and it is usually resolved by conventional conversion to the mono-ester of succinic or phthalic acid. This hydrogen succinate or phthalate is then converted into diastereomeric salt by contact with optically active bases as described herein.

The pre-steps include:
1. Treat the racemic alcohol with 1×1 molar ratio of phthalic anhydride and greater than 1×1 molar ratio of pyridine. It is permitted to use 1×1 molar ratio of succinic anhydride instead of the pyridine.
2. Heat the mixture to between about 80 to 100° C. for 2 hrs.
3. Cool the mixture to ambient temperature and then quench with ice water containing sufficient sulfuric acid to make the whole mixture acidic, i.e., a pH less than 7. This mixture will be the hydrogen phthalate, either in the form of oil or as a crystalline solid. If the mixture is oil, treat it with acetone and/or use conventional crystal initiation techniques as necessary to crystallize it.
4. Filter, wash and then dry the mixture. The product of the hydrogen phthalate with free having a carboxyl function.
5. Use the kits B1, B2, B3, etc. as described above.

Pre-Processing for Amino Acids (Amphoteric Racemate)

Amino acids exist in Zwitter ion (dual charged) structure. A synthetic amino acid is primarily resolved using one of the following two types of methods:
1. By protecting the carboxyl group (and freeing the amino group), usually using esterification, or
2. By protecting the amino group (and freeing the carboxyl group), usually using formylation.

### Protection of Carboxylic Group Using Esterification:

The carboxyl end of the molecule is protected by standard esterification using mild base and an alcohol followed by diastereomeric salt formation of the free amine function and needs screening kits made up of chiral acids. Many racemic alpha-amino acids have been successfully resolved by preparing the corresponding isobutyl ester or benzyl ester.

Steps:
1. Add a sufficient amount of dilute HCl to the racemate to dissolve it and adjust the pH to 3.
2. Cool the mixture to between about 0 to 2° C.
3. Esterify by adding (1:1.2 ratio) of isobutyl ester or benzyl ester.
4. Heat the mixture to 100° C. and then cool it to 0 to 5° C.
5. Decrease the acidity to pH 7 by adding NaOH.
6. Use the kits A1, A2, A3, etc. as described above to obtain the diastereomeric salt.
7. After having identified the ideal candidate vial containing crystals, then remove the ester group introduced in step 3 under mild acid hydrolysis conditions and verify that no racemization has occurred.

### Protection of Amino Group Using Formylation:

The carboxylic group is then screened with the amines kits (B1, B2, B3, etc.). After having identified the ideal candidate vial, one then removes the formyl group under mild hydrolysis conditions and verifies that no racemization has occurred.

Steps:
1. Add a sufficient amount of 1N NaOH solution to the racemate to dissolve it and bring the pH to 10.
2. Cool the mixture to 0 to 2° C.
3. Formylate by adding (1:1.2 ratio) of triethyl orthoformate.
4. Heat the mixture to 100° C. and then cool it to 0 to 5° C.
5. Increase the acidity to pH 4 by adding hydrochloric or sulfuric acid.
6. Use the kits B1, B2, B3, etc. as described above.
7. After having identified the ideal candidate vial, one should then remove the formyl group introduced in step 3 under mild acid hydrolysis conditions and verify that no racemization has occurred

### Preprocessing for Aldehydes and Ketones

In order to be resolved by salt formation, aldehydes and ketones must be transformed into either acidic or basic derivatives.

### Acidic Derivatives:

Reagents such as 4-sulfonylphenylhydrazine, 4-(4-carboxyphenyl) semicarbazide, 4-hydrazinobenzoic acids (para/meta), oxalic acid monohydrazide is used. These salts are then be resolved by chiral bases.

Steps:
1. Treat the racemic aldehyde or ketone in minimum amount of methanol
2. Cool the mixture to 0° to 5° C.
3. Add one of the above-cited reagents. (The result is a crystalline protected amino acid.)
4. Isolate the protected amino acid using filtration or centrifugation.
5. Use the kits B1, B2, B3, etc. as described above

### Basic Derivatives:

A carbonyl can be converted into enamine using tertiary amines, which enamine is then resolved by chiral acids. Alternatively, carbonyl is treated with bisulphite salts of chiral amines, and resulting diastereomers are separated by crystallization.

Steps:
1. Treat the racemic aldehyde or ketone in minimum amount of methanol.
2. Cool the mixture to 0 to 5° C.
3. Add tertiary amine like pyrrolidine or piperidine. Alternatively one adds sodium bisulphite. (The result is a crystalline protected amino acid.)
4. Isolate the protected amino acid using filtration or centrifugation.
5. Use the kits B1, B2, B3, etc. as described above.

### Diastereomeric Crystallization Technique

Chirally-pure isomers are obtained through a variety of techniques. The most commonly used one is the classic resolution by diastereomeric crystallization. Because of its easy adoption in a manufacturing setting, most companies try this approach first; and then use other approaches only if this one fails. Currently, over 65% of all chiral products are developed using this technique.

The primary reasons for preferring diastereomeric crystallization in manufacturing are economic, that is:
a) It is easier and therefore cheaper to build up the racemate needed for resolution methods than it is to create pure isomers using the synthetic technologies.
b) Among the resolution techniques available, resolution by diastereomeric crystallization is less time and temperature sensitive and less complex.
c) The equipment for doing diastereomeric crystallization is more likely to already exist in manufacturing installations.
d) Racemization in connection with diastereomeric crystallization ultimately produces a high yield of the enantiomer much more cheaply than the other resolution or synthetic procedures. (Racemization is the process of repeatedly reprocessing the “waste” portion of the resulting products; each subsequent pass yielding additional good product)
e) Resolution by diastereomeric crystallization is also generally superior to enzymatic resolution in that it usually yields a product of higher enantiomeric purity and both isomers are separated. In enzymatic resolution, one isomer is usually destroyed. Also, enzyme resolution generally does not yield a highly pure (ee) isomer and thus one needs to utilize diastereomeric crystallization as the last step.

### Resolving Agents

A classical resolving agent is a chiral acid or base (optically active isomer, enantiomer), which has a propensity to form a crystalline diastereomer when combined with a racemic base or acid. Some requirements of an ideal resolving agent include:

1. Proximity of stereogenic centers,

2. Rigid structure,

3. Must have strong acid or base characteristics,

4. Must have chemical and optical stability,

5. Both enantiomers must be available and recyclable, and

6. Must be availability in bulk quantities at relatively low price.

Amines and cinchonal alkaloids found typically in natural products meet these requirements and are used most often.

### Resolution of Different Materials

For resolving carboxylic acids one usually forms salts with optically active amines. On the other hand, for resolving amine: one uses enantiomeric pure acids such as tartaric acid, malic acid and mandelic acid.

To resolve neutral compounds, one prepares covalent diastereomeric derivatives. e.g. with alcohols, one forms monophthalate, succinate or ester; while with ketones, one forms the corresponding hydrazones.

### Resolution of Amino Acids (Amphoteric Racemates)

Amphoteric racemates have both acidic and basic characteristics, e.g., in aspartic acid, there are two carboxylate groups for one amine group. The compound is resolved as a simple acid or base. For compounds having one carboxyl and amino group each, one of the functional group must be functionalized.

### Resolution of Neutral Compounds

If resolution of a neutral compound by salt formation is intended, the compound must first be transformed to a derivative containing an acidic or basic group. Resolution by derivatization is typical for alcohols, aldehydes and ketones. Alcohols are almost exclusively transformed to their monophthaletes or succinates. Usually phthalates (phthalic or 3-nitrophthalic anhydride) or succinic anhydride for succinates are used.

The inherent low yields of resolution are increased to nearly 100% using various techniques. The best resolutions are those in which the undesirable enantiomer is later racemized and recycled to produce overall yields close to 100%.

The following examples are provided for description and explanation only. They are not to be construed to be limiting in any way.

### EXAMPLES

### General

The solvents are available from commercial sources, usually as reagent-grade and used without further modification.

The acid and base resolving agents are available from commercial sources and are used without further purification.

The reagents to transform a “neutral” precursor compound to a useful derivative are available from commercial sources and are used without purification.

The kits (tray and tube combinations) are available from the inventor as ChiroSolve, Inc. of Cupertino, Calif. (See http//:www.chirosolve.com)

Commercial chemical suppliers include, but are not limited to, Aldrich Chemical, Milwaukee, Wis., MP Biomedicals, Irvine, Calif., etc.

Additional sources are located in Chemical Sources USA, published annually by Chemical Sources International, Inc. of Clemson, S.C., 29633.

Solvent and chemical commercial sources are also found in Chemical Sources, USA at www.chemsources.com.

The tubes or containers, stoppers, film, etc. are commercially available from chemical supply houses such as E & C Scientific, Inc.; Matrix Technologies, Inc., Hudson, N.H.; Abgene, Inc., Rochester, N.Y.; TomTec, Inc., Hamden, Conn.; and Micronic MA, McMurray, Pa. The object is a commercial film having adhesive or quasi-adhesive properties. Usually it is a polymer, aluminum, and/or combinations thereof. The sheet is useful to retain solvents and resolving agents prior to use. The commercial septums perform the same function for the individual containers.

The tubes or containers with the bar code or alphanumeric code labels are made from commercially available makers for example, Matrix, Inc., Abgene, Inc.

### Example 1

### Screening of Racemic Acid Using Chirosolv® Kit

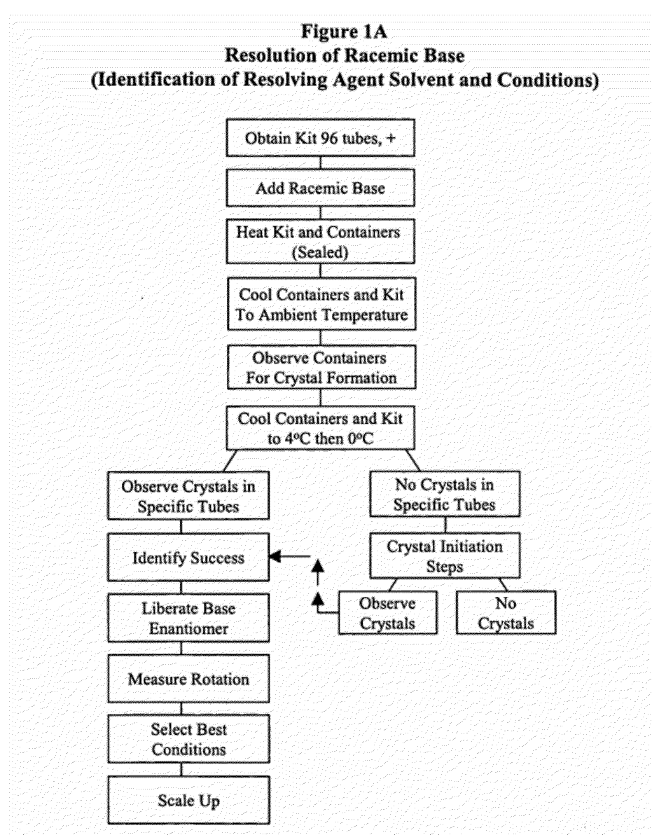

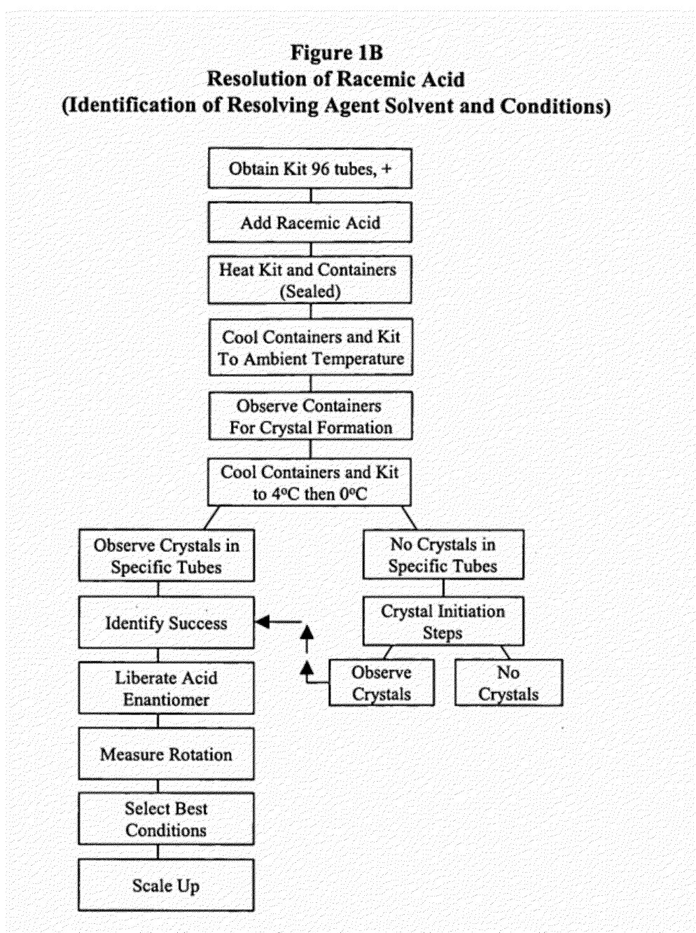

(This Procedure Corresponds in General to FIG. 1b.)
1. Use kits B1, B2, B3 B4 or combination thereof.
2. Remove the lid of the kit(s).
3. Determine if the unknown racemate acid is solid/powder.
   a) If yes, remove the seal of the kit and dispense about 0.001 to 0.03 mmol of unknown racemate into each container of the kit. Cover the containers with additional seal/rubber septa provided. Go to step 4.
   b) If no, dispense about 0.001 to 0.03 mmol of the liquid racemate into each container.
4. Heat the kit and containers and the mixture to 80° C., or until the mixture becomes homogeneous (up to 100° C.).
5. Optionally agitate the kit to encourage homogenization.
6. Cool the kit with containers and mixtures to ambient temperature.
7. Determine if any crystals formed.
   a) If yes, select the containers with crystals, close them with additional rubber septum provided and set them aside for further analysis. Go to step 11.
   b) If no, proceed to step 8.
8. Cool the kit with containers and mixtures further to 4° C. and then to 0° C.
9. Optionally use crystal initiation technique to encourage crystal formation.
10. Determine if any crystals formed.
   a) If yes, select the containers with crystals, close them with additional rubber septum provided and set them aside for further analysis. Go to step 11.
   b) If no, discard the containers without the crystals and exit.
11. Note the identification marking (bar code or alphanumeric code) of the kit as well as the individual containers that are to be analyzed further.
12. Analyze the crystals of each container selected separately after liberating enantiomers from the respective diastereomeric salts for specific rotation measurement using sodium or mercury light.
13. Select one or two containers out of all containers in step 12 with crystals that have maximum optical purity. These are the optimal resolving agent and solvent combinations for the given racemate.
14. Evaluate selected crystals in step 13 with its mother liquor for scale-up optimization.

### Example 2

### Resolution of Racemic Bases Using Chirosolv® Kit

(This Procedure Corresponds in General to FIG. 1a.)
1. Use kits A1, A2, A3 A4 or combination thereof.
2. Remove the lid of the kit(s).
3. Determine if the unknown racemate base is solid/powder.
   a) If yes, remove the seal of the kit and dispense about 0.001 to 0.03 mmol of unknown racemate into each container of the kit. Cover the containers with additional seal/rubber septa provided. Go to step 4.
   b) If no, dispense about 0.001 to 0.03 mmol of the liquid racemate into each container.
4. Heat the kit and containers and the mixture to 80° C., or until the mixture becomes homogeneous (up to 100° C.).
5. Optionally agitate the kit to encourage homogenization.
6. Cool the kit with containers and mixtures to ambient temperature.
7. Determine if any crystals formed.
   a) If yes, select the containers with crystals, close them with additional rubber septum provided and set them aside for further analysis. Go to step 11.
   b) If no, proceed to step 8.
8. Cool the kit with containers and mixtures further to 4° C. and then to 0° C.
9. Optionally use crystal initiation technique to encourage crystal formation.
10. Determine if any crystal formed.
   a) If yes, select the containers with crystals, close them with additional rubber septum provided and set them aside for further analysis. Go to step 11.
   b) If no, discard the containers without the crystals and exit.
11. Note the identification marking (bar code or alphanumeric code) of the kit as well as the individual containers that are to be analyzed further.
12. Analyze the crystals of each container selected separately after liberating enantiomers from the respective diastereomeric salts for specific rotation measurement using sodium or mercury light.
13. Select one or two containers out of all containers in step 12 with crystals that have the maximum optical purity. These are the optimal resolving agent and solvent combinations for the given racemate.
14. Evaluate selected crystals in step 13 with its mother liquor for scale-up optimization.

Our statistical screening resolving agents and optimization of resolution conditions are systematically studied by the present description in the chiral kit experiment.

Typical results of screening experiments are given below:

TABLE 4
Resolution of (±) Phenylpropionic acid (Hydratropic acid)

| Amines | Ethanol | 96EtOH | MeOH | 80MeOH | Eacetatate | 70IPA | 99IPA | 1-butanol |
|---|---|---|---|---|---|---|---|---|
| Phenethyl | Oil | Oil | Oil | Oil | α = −5.1 | Oil | Oil | α = 0 |
| Megluca- | α¹ = +4.0 | α = +3.0 | Oil | Oil | Oil | Oil | Oil | Oil |
| Strychnin | α = +7.0 | α = +7.5 | α = +5.2 | α = +5.5 | Oil | Oil | Oil | Oil |
| Quinidin | α = −3.0 | α = −5.2 | Oil | Oil | α = +2.0 | α = 0 | Oil | Oil |
| Quinin | α = +5.0 | α = +4.5 | Oil | α = +2.0 | Oil | Oil | Oil | Oil |
| Brucine | Oil | Oil | Oil | Oil | α = −1.2 | Oil | Oil | Oil |

αethanol(C = 0.1)
¹= Has to be cooled to 4° C.
Note:
All cells with oil indicates negative test that you should discard (after usual crystallization efforts)
Above table shows that the strychnine in 96% Ethanol is ideal system for (+) isomer, while quinidine in 96% Ethanol would be good system for (−) isomer.

TABLE 5
Resolution of (±) Phenylpropionic acid (Hydratropic acid)

| Acids | Ethanol | 96EtOH | MeOH | 80MeOH | Eacetatate | 70IPA | 99IPA | 1-butanol |
|---|---|---|---|---|---|---|---|---|
| Tartaric | α = 0 | α = −0.5 | α¹ = +5.0 | Oil | NA² | Oil | α¹ = 0 | Oil |
| Pyrogluta | Oil | Oil | Oil | Oil | NA² | α = +15.5 | Oil | Oil |
| Malic | α = +6.0 | α = +10.0 | α = −1.0 | Oil | NA² | α = +6.5 | Oil | α¹ = −5.0 |
| Mandelic | Oil | Oil | Oil | Oil | Oil | Oil | Oil | Oil |
| Dtolytart | NA² | NA² | α = +14.5 | NA² | NA² | NA² | NA² | Oil |
| Camphor | Oil | Oil | Oil | Oil | Oil | Oil | Oil | Oil |

¹= Little solvent was evaporated to yield crystals
NA² = Did not go in solution even at 80° C.
α = Neat

As evident from above data (+) isomer of amine, pyroglutamic acid in 70% IPA is ideal system, for (−)isomer, malic acid in 1-butanol is the system of choice. The literature shows malic acid in ethanol was used to resolve the racemic amine.

There are a few new developments in the field: one is by a Roche group in the United Kingdom, which has used differential scanning calorimetry as a means of to identify diastereomeric salts with a clear eutetic composition that is needed for effective resolution (Ref. 5). They also utilized robots to synthesize diastereomeric salts and facilitated data analysis by developing resolution package. In process called “Dutch Resolution” a family of resolving agents is being used instead of single agent, for example tartaric acid family composed of dibenzoyltartaric acid, ditolyltartaric acid and tartaric acid. According to the authors when such a mixture is added to a solution of a racemic substrate, a crystalline salt usually precipitates immediately. In most cases the substrate contained in the precipitated salt is resolved to about 90-98% ee (Ref. 6).

### Further Case Studies for Developing Pure Enantiomer

### N-benzyl-1-(4-methylphenyl)propan-2-amine

The enantiomer preparation kit described herein was used with N-benzyl-1-(4-methylphenyl)propan-2-amine (racemate had 70% 5 and 30% R isomer). Goal was to obtain over 90% purity for R isomer. This racemate was resolved by Di-tolyltarataric (+) acid in 100% IPA. After two re-crystallizations 30% 5 and 70% R was obtained. Additional two re-crystallizations yielded over 90% enrichment in R. The yield was over 90% of theoretical value

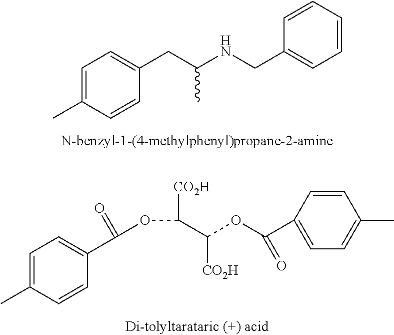

### N-Benzyl-(4-benzylphenyl)propane-2-amine

The enantiomer preparation kit described herein was used with N-Benzyl-(4-benzylphenyl)propane-2-amine (racemate had 50% 5 and 50% R). Goal was to obtain over 90% purity for R isomer. This racemate was resolved by S-Acetylmandelic acid (+) in 90% IPA. After two re-crystallizations, 80% R and 20% S, was obtained with yield over 80% of theoretical value. One more re-crystallization yielded over 92% purity in R.

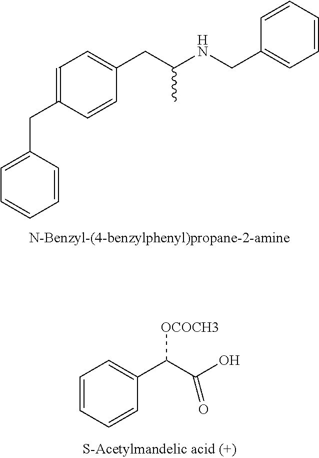

Results for the case studies with N-benzyl-1-(4-methylphenyl)propan-2-amine and -Benzyl-(4-benzylphenyl)propane-2-amine are described in Table 6 below:

TABLE 6

| | | | | | Amount |
|---|---|---|---|---|---|
| | Desired | | | | of pure |
| | isomer | Starting | Amount | # recrys- | enantio- |
| Racemate | purity | material | recovered | tallization | mer |
| N-Benzyl-(4- | R isomer, | 1.046 | 1.009 gm | 4 | 360 mg |
| methylphenyl) | >90% purity | gm | (96%) | | |
| propane- | | | | | |
| 2-amine | | | | | |
| N-benzyl-1- | R isomer, | 1.931 | 1.888 gm | 3 | 697 mg |
| (4-benzyl- | >90% purity | gm | (97%) | | |
| phenyl)- | | | | | |
| propane- | | | | | |
| 2-amine | | | | | |

Having now described a few embodiments of the invention, it should be apparent to those skilled in the art that the foregoing is merely illustrative and not limiting, having been presented by way of example only. Numerous modifications and other embodiments are within the scope of one of ordinary skill in the art and are contemplated as falling within the scope of the invention and any equivalent thereto. It can be appreciated that variations to the present invention would be readily apparent to those skilled in the art, and the present invention is intended to include those alternatives. Further, since numerous modifications will readily occur to those skilled in the art, it is not desired to limit the invention to the exact construction and operation illustrated and described, and accordingly, all suitable modifications and equivalents may be resorted to, falling within the scope of the invention.

1-6. (canceled)

7. A method of crystallization of diastereomeric salts, said method comprising the steps of:
(a) identifying at least one of a plurality of resolving agents and at least one of a plurality of solvents for separating enantiomeric isomers from a racemate, wherein the identification of the at least one of a plurality of resolving agents and the at least one of a plurality of solvents comprises the steps of:
(i) adding about 0.001 to about 0.05 mmol of the at least one resolving agent and about 50 to about 200 microliter of the at least one solvent to each of a plurality of containers arranged in an array,
(ii) adding to each of the plurality of containers a predetermined quantity of the racemate to form a unique combination in each container of the at least one resolving agent, the at least one solvent and the racemate, wherein the ratio between the racemate and the resolving agent is between 1:1 to 1:0.25,
(iii) heating the combination in each of the plurality of containers to a predetermined first temperature, wherein said predetermined first temperature is less than 80° C.,
(iv) cooling the heated combination in each of the plurality of containers to a predetermined second temperature, wherein said predetermined second temperature is greater than −4° C.,
(v) determining whether diastereomeric crystals are formed in each of the plurality of containers,
(vi) selecting at least one of the plurality of containers comprising the diastereomeric crystals,
(vii) optionally separating the diastereoisometric crystals from the combination in each of the plurality of selected containers,
(viii) optionally isolating an isomer from the separated diastereomeric crystals in each of the plurality of selected containers,
(ix) evaluating the purity of the diastereoisometric crystals or of the isomer in each of the plurality of containers,
(x) identifying the container(s) from the plurality of containers corresponding to the isomer having a highest purity, and
(xi) selecting the combination(s) associated with the identified container(s) corresponding to the isomer having the highest purity;
(b) recovering the racemate from the combination(s) in each of the plurality of containers, wherein the separating step comprises:
(i) optionally heating the first combination of the at least one resolving agent, the at least one solvent and the racemate in each of the plurality of containers to a temperature of less than 80° C.,
(ii) collecting the heated combination(s) from each of the plurality of containers in a plurality of first collection containers,
(iii) adding a recovery solution to the heated combination(s) of the at least one resolving agent, the at least one solvent and the racemate in the plurality of first collection containers to form a plurality of second combinations, wherein the recovery solution has a predetermined pH value,
(iv) mixing each of the plurality of second combinations in each of the plurality of first collection containers,
(v) transferring the second combinations from the plurality of first collection containers into a separatory funnel and letting stand until two liquid layers form, wherein the first layer comprises the resolving agents and solvents and the second layer comprises the racemate and solvents,
(vi) extracting the second layer from said separatory funnel to a plurality of second collection containers, and
(vii) evaporating the at least one solvent from each of the plurality of second collection containers to recover the racemate; and
(c) purifying the isolated isomers to a predetermined purity, wherein the purification comprises the steps of:
(i) identifying stoichiometric ratio of the at least one resolving agent, the at least one solvent and the racemate from the combination(s) associated with the identified container(s) corresponding to the isomer having the highest purity of step (a)(xii);
(ii) preparing a plurality of third combinations comprising a predetermined amount of the at least one resolving agent, the at least one solvent and the racemate having the identified stoichiometric ratio, wherein each of the plurality of third combinations is prepared in the plurality of second collection containers,
(iii) heating the plurality of third combinations in each of the plurality of second collection containers to the predetermined temperature,
(iv) stirring the heated plurality of third combinations in each of the plurality of second collection containers to form a homogenous mixture,
(v) cooling the plurality of third combinations in each of the plurality of second collection containers to a predetermined third temperature,
(vi) initiating formation of crystals of enantiomeric salt in each of the plurality of second collection containers,
(vii) filtering the plurality of third combinations in each of the plurality of second collection containers to separate the crystals of enantiomeric salt from a filtrate, wherein the filtrate comprises the at least one resolving agent, the at least one solvent and the racemate, wherein the filtrate from in each of the plurality of second collection containers is collected in a plurality of third collection containers,
(viii) measuring the purity of the separated crystals of the enantiomeric salt from each of the plurality of second collection containers,
(ix) comparing the measured purity of the separated crystals of the enantiomeric salt from each of the plurality of second collection containers to the predetermined purity,
(x) when the measured purity in (c)(viii) is less than the predetermined purity, adding the separated crystals from step (c)(vii) to the plurality of second collection containers,
(xi) heating a predetermined quantity of the at least one solvent to about its boiling point,
(xii) adding the heated solvent from step (c)(xi) to the plurality of second collection containers,
(xiii) stirring the contents in each of the plurality of second collection containers,
(xiv) cooling the contents in each of the plurality of second collection containers to a predetermined fourth temperature,
(xv) initiating formation of crystals of enantiomeric salt in each of the plurality of second collection containers,
(xvi) collecting the crystals of enantiomeric salt in each of the plurality of second collection containers,
(xvii) repeating steps (c)(vii)-(c)(xvi) until the measured purity of the separated crystals of the enantiomeric salt in at least one of the plurality of second collection containers is substantially the same as the predetermined purity, and
(xviii) Optionally repeat steps (b) and (c) to prepare alternative enantiomers having measured purity.

8. The method of claim 7, wherein the step (c) of purifying the isolated isomers further comprises treating the enantiomeric salt with an acid having a pH of at most 3 to liberate the enantiomer from the enantiomeric salt, wherein the enantiomeric salt is an acid.

9. The method of claim 8, wherein the acid is selected from group comprising citric acid and hydrochloric acid.

10. The method of claim 7, wherein the step (c) of purifying the isolated isomers further comprises treating the enantiomeric salt with a base having a pH of at least 9 to liberate the enantiomer from the enantiomeric salt, wherein the enantiomeric salt is a base.

11. The method of claim 10, wherein the base is selected from group comprising sodium (or potassium) carbonate, potassium carbonate, sodium bicarbonate, potassium bicarbonate, sodium hydroxide, and potassium hydroxide.

12. The method of claim 7, wherein the step (c) of purifying the isolated isomers further comprises recovering the racemate from filterate collected in the plurality of third collection containers.

13. The method of claim 7, wherein the step of identifying the at least one of a plurality of resolving agents and the at least one of a plurality of solvents further comprises agitating the combination from about 5 minutes to about 24 hours.

14. The method of claim 7, wherein the formation of diastereomeric crystals in each of the plurality of containers is determined by optical measurements.

15. The method of claim 7, wherein when the racemate is acidic the recovery solution has a pH of at most 4.

16. The method of claim 7, wherein when the racemate is base the recovery solution has a pH of at least 9.

17. The method of claim 7, wherein the first layer in the separatory funnel comprises the resolving agents, water and the at least one solvent of the plurality of solvents, wherein the second layer in the separatory funnel comprises the racemate and at least one solvent of the plurality of solvents, wherein the first layer is above the second layer.

18. The method of claim 7, wherein the each container in the array of containers having optionally a barcode marking or alphanumeric marking, each of said containers ranging in size from 0.5 milliliter to 4 milliliter, each container being optionally sealed with sealant(s) or stopper(s) to avoid loss of said solvent, each of said sealant(s) or stopper(s) being stable at temperatures between −20° C. to 120° C.

19. The method of claim 7, wherein the resolving agent is chemically and optically stable, has optical purity equal to or higher than the target purity of the resultant enantiomer, and does not racemize under the conditions of said method.

20. The method of claim 7, wherein the initiation of crystal formation comprises scratching the inside corners of the second collection container.

21. The method of claim 7, wherein said at least one solvent is selected from the group consisting of 90 percent acetone, methyl ethyl ketone (2-butanone), i-butanol, 2-propanol, 90 percent 2-propanol, methanol, 80 percent methanol, ethanol, 96 percent ethanol, water, 1-propanol, 85 percent 1-propanol, acetonitrile, ethyl acetate, dichloromethane, chloroform, p-dioxane, methyl-t-butyl ether, toluene and tetrahydrofuran;

22. The method of claim 7, wherein said at least one resolving agent, is at least one of a chirally pure acid or a chirally pure base and is selected from the group consisting of tartaric acid, pyroglutamic acid, di-p-tolulo-tartaric acid, mandelic acid, malic acid, camphorsulphonic acid, dibenzoyl-tartaric acid, deoxycholic acid (+), camphoric acid (+), quinic acid (−), aspartic acid (+), glutamic acid, 1,3,4,6-diisopropylidine-2-ketogluconic acid (−), acetylmandelic acid, N-acetyl-1-hydroxyproline, N-acetyl-1-leucine, acetyl-3-mercapto-2-methylpropionic acid, 3-acetylmercapto-2-methylpropionyl-1-proline, N-acetyl-D-3-(2-naphthyl)-alanine, (R)-acetylthio-2-methylpropionyl chloride, N-acetyl-1-phenylalanine, N-acetyl-1-tyrosinamide, D-alanine, 1-aminoadipic acid, (R)-2-aminobutyric acid, (1R,4S)-4-aminocyclopent-2-ene-1-carboxylic acid, (1S,4R)-4-aminocyclopent-2-ene-1-carboxylic acid, S-2-amino-3,3-dimethylbutyric acid, 1-tert-leucine, 1,2-amino-2-methyl-3-(3′,4′-dimethoxyphenyl)-propionitrile HCl, 1-2-amino-2-methyl-3-(3′,4′-dihydroxyphenyl)-propionic acid, (R)-2-amino-4-phenylbutane, D-arginine, D-aspartic acid, D-2-azidophenylacetic acid, D-2-azidophenylacetyl chloride, (1S,2R)-cis-2-benzamido-cyclohexane-carboxylic acid, (1R,2S)-cis-2-benzamido-cyclohexane-carboxylic acid, benzyl-(R) & (S)-mandelate, benzyl-2-tosyloxypropionate, N-2-BOC-D-alanine, N-2-BOC-1-aminoadipic acid, 3-(R)-BOC-aminocyclopent-4-ene-1-(S)-carboxylic acid, 3-(S)-BOC-aminocyclopent-4-ene-1-(R)-carboxylic acid, N-2-BOC-D-arginine hydrochloride, N-2-BOC-D-aspartic acid, N-2-BOC-3-(4-biphenyl)alanine, N-2-BOC-N-6-CBZ-D-lysine, N-2-BOC-3-(4-chlorophenyl)-alanine, N-2-BOC-cyclohexylalanine, N-2-BOC-1-cyclohexylalanine methyl ester, N-2-BOC-3,3-diphenylalanine, N-2-BOC-3-(4-fluorophenyl)-alanine, N-2-BOC-D-glutamic acid 1-benzyl ester, N-2-BOC-D-histidine, N-2-BOC-3-(4-iodophenyl)-alanine, N-3-BOC-D-leucine, N-3-BOC-1-tert-leucine DCHA salt, (1S)-camphanic acid, (1R)-camphorsulfonic acid, (1S)-camphorsulfonic acid, 2-methylbenzylamine, N-2-BOC-D-methionine, N-2-BOC-3-(1′-naphtyl)alanine, N-2-BOC-3-(2′-naphtyl)alanine, N-2-BOC-3-(4′-nitrophenyl)alanine, N-2-BOC-1-octahydroindole-2-carboxylic acid, N-2-BOC-3-(pentafluorophenyl)-alanine, N-2-BOC-D-phenylalanine, N-BOC-D-proline, N-1-BOC-D-3-(2′-pyridyl)alanine, N-2-BOC-1-3-(2′-pyridyl)alanine, N-2-BOC-D-3-(3-pyridyl)alanine, N-1-BOC-1-3-(3′-pyridyl)alanine, N-2-BOC-D-serine, N-BOC-1,2,3,4-tetrahydroisoquinoline-(3R)-carboxylic acid, N-BOC-1,2,3,4-tetrahydroisoquinoline-(3S)-carboxylic acid, N-2-BOC-3-(4′-thiazolyl)alanine, N-BOC-D-threonine, N-2-BOC-N-8-tosyl-D-arginine, N-2-BOC-D-tryptophan, N-2-BOC-D-tyrosine, N-2-BOC-D-tyrosine methyl ester, N-2-BOC-D-valine, 2-bromobutyric acid, 2-bromohexadecanoic acid, (R)-2-bromo-2-phenylacetic acid, 2-bromopropionic acid, butyl-(S)-2-chloropropionate, (2R,3S)-butyl-2,3-epoxybutyrate, (R)-butyl-2,3-epoxybutyrate, (S)-tert-butyl-3-hydroxybutyrate, (S)-butyl-lactate, N-butyl-(R)-2-methyl-2-hydrazino-3-(3′-methoxy-4′-hydroxyphenyl)-propionate, N-CBZ-D-alanine, N-CBZ-D-arginine, N-CBZ-D-aspartic acid, N-CBZ-O-tert-butyl-D-serine, CBZ-1-cyclohexylalanine, N-CBZ-D-glutamic acid, N-CBZ-D-histidine, N-CBZ-D-leucine, N-CBZ-1-tert-leucine DCHA salt, N-CBZ-D-methionine, N-2-CBZ-D-3-(2′-naphthyl)alanine, N-2-CBZ-ornithine, N-2-CBZ-D-phenylalanine, N-2-CBZ-D-proline, N-2-CBZ-D-serine, N-2-CBZ-D-threonine, N-2-CBZ-D-tryptophan, N-2-CBZ-D-tyrosine, N-2-CBZ-D-valine, (R)-2-chlorobutyric acid, 3-chloromandelic acid, 4-chloromandelic acid, 1-((S)-3-chloro-2-methylpropionyl)-1-proline, (R)-2-(4′-chlorophenoxy)-propionic acid, 3-(4′-chlorophenyl)alanine, 2-(4′-chlorophenyl)-3-phenylpropionic acid, chlorophos, 2-chloropropionic acid, (S)-2-chloropropionic acid sodium salt (50 percent solution), cyclohexylalanine, cyclohexylglycine, cyclophos, D-cysteine, D-cysteine hydrochloride monohydrate, D-cysteine, dibenzoyl-tartaric acid, 1-3-(3′,4′-dichlorophenyl)-alanine, diethyl-1-tartrate, D-1-dihydrophenylglycine, D-1-dihydrophenylglycine chloride hydrochloride, D-(3′,4′-dihydroxy)-1-phenylglycine, diisopropyl-tartrate, dimethyl-tartrate, 2-3-diphenylpropionic acid, di-p-toluoyl-tartaric acid, ethyl-(R)-2-(N-acetylamino)-2,4-dimethylpentanoate, ethyl-(R)-2-(N-acetylamino)-2-methyl-3-phenylpropionate, ethyl-4-bromo-3-hydroxybutyrate, ethyl-4-chloro-3-hydroxybutyrate, ethyl-(S)-2-chloropropionate, ethyl-2-3-dihydroxybutyrate, ethyl-2-3-dihydroxy-3-phenylpropionate, (R)-ethyl-3-hydroxybutyrate, ethyl-2-hydroxy-2-phenylacetate, ethyl-(R)-2-hydroxy-4-phenybutyrate, ethyl-3-hydroxy-3-phenylpropionate, (R)-ethyl-4-iodo-3-hydroxybutyrate, N-(1-phenylethyl)-phtalimide, D-phenylglycine, N,N,N′,N′-tetramethyl-tartaric acid, thiazolidine-4-carboxylic acid, 3-(2-thienyl)-alanine, D-allo-threonine, valine, N-methylglucamine (−), α-methylbenzylamine, cinochonidine (−), ephedrine (−), hydroquinidine (+), N-benzyl-α-methylbenzylamine, brucine (−), strychnine (−), pseudoephedrine (+), qunidine, quinine (−), cinchonine (+), threo 2-amino-1-(p-nitrophenyl)-1,3-propanediol, 2-amino-1-butanol, methylephedrine (−), α-1-naphthylethyl amine, dehydroabietyl amine, 2-amino-1-phenyl-1,3-propanediol, D-alaninamide, 2-amino-1-propanol, 2-aminobutanol, erythro-2-amino-1,2-diphenylethanol, (S)-1-aminoindane, cis-(1S,2R)aminoindan-2-ol, 1-amino-2-(methoxymethyl)-pyrrolidine, 2-amino-3-methyl-1-butanol, 2-amino-3-methyl-1-pentanol-isoleucinol, 2-amino-4-methyl-1-pentanol-leucinol, 2-amino-1-[4′-(methylthio)-phenyl]-1,3-propanediol, 2-amino-1-phenylethanol, 1-amino-2-propanol, 1-aminotetralin and N-propyl derivative, 2-aminotetralin and N-propyl derivative, N-benzyl-3-aminopyrrolidine, benzyl-benzyl amine, benzyl-4-chlorobenzylamine, cis-N-benzyl-2-(hydroxymethyl)cyclohexylamine, N-benzyl-3-hydroxypyrrolidine, N-benzyl-2-methylbenzylamine, N-benzylamine-methylbenzylamine hydrochloride, 2-benzyl-2-methylbenzylamine, 2-benzyl-3′-methylbenzylamine, 2-benzyl-4′-methylbenzylamine, N-benzyl-1-(1′-naphthyl)ethylamine hydrochloride, bis(methoxymethyl)pyrrolidine, bis{1-[1-naphthyl]ethyl}amine hydrochloride, bis(1-phenylethyl)amine hydrochloride, N,N-bis-[1-phenylethyl]phthalamic acid, N-2-BOC-cyclohexylglycine, BOC-isoleucinol, BOC-phenylalaminol, BOC-prolinol, N-butyl-2-amino-2-methyl-3-(3′,4′-dihydroxyphenyl)-propionate, CBZ-1-cyclohexylalaminol, N-2-CBZ-D-3-(1′naphthyl)alaminol, N-2-CBZ-D-3-(2′naphthyl)alaminol, N-1-phenylalaminol, 2-(2′-chlorobenzyl)benzyl-amine, 2-(3′-chlorobenzyl)benzyl-amine, 2-(4′-chlorobenzyl)benzylamine, (S)-cyclohexylalaminol, 1,2-diaminocyclohexane, (S)-2,6-diamino-1-hexanol (1-lysinol), 1,2-diaminopropane, 2,2-dibenzyl-2-hydroxy-1-methylethylamine, N,N-dibenzylphenylalaminol, N-(3,4-dimethoxybenzyl)-1-phenylethylamine, 3,3-dimethyl-2-aminobutane, N,N-dimethyl-1-methylbenzylamine, N,N-dimethyl-2-(1′-naphthyl)ethylamine, N-(3′,4′-dinitrobenzoyl)-2-methylbenzylamine, N-(3′,5′-dibenzoyl)-1-(1-naphthyl)ethylamine, 1,2-diphenyl-1,2-ethanediamine, 2,2-diphenyl-2-hydroxy-1-methylethylamine, N,N′-ditosyl-1,2-diphenyl-1,2-ethanediamine, 2,2-diphenyl-2-hydroxy-1-methylethylamine, N,N′-ditosyl-1,2′-diphenyl-1,2-ethanediamine, diphenylvalinol, diphenylprolinol, ethyl-(R)-2-amino-2-methyl-3(3′,4′-dimethoxyphenyl)propionate, ethyl (R)-2-amino-2-methyl-3-phenylpropionate, 3-hydroxypyrrolidine, 3-hydroxypyrrolidine HCl, isopropyl-2-methylbenzylamine, 1-tert-leucinol, 1-tert-leucinol hydrochloride, 1-methioniol, 5-methoxy-2-aminotetralin, N-propyl-5-methoxy-2-aminotetralin, 6-methoxy-2-aminotetralin and N-propyl-6-methoxy-2-aminotetralin, 7-methoxy-2-aminotetralin and N-propyl, 8-methoxy-2-aminotetralin and N-propyl, (S)-2-(methoxymethyl)pyrrolidine, (S)-2-(methylamino)propiophenone, D-N-methylamphetamine, 2-(4′-methylbenzyl)benzylamine, 2-(4′methylbenzyl)-N′N′-dimethylbenzylamine, 2-(4′-methylbenzyl)-N-hydroxyethyl-benzylamine, 2-methyl-3′-bromobenzylamine, 2-methyl-4′-bromobenzylamine, 2-methyl-4′-bromobenzylamine hydrochloride, 2-methyl-4′-chlorobenzylamine, 2-methyl-2′-methoxybenzylamine, 2-methyl-3′-methoxybenzylamine, 1-methyl-3′-methoxybenzylamine, 2-methyl-4′-methoxybenzylamine, 2-methyl-4′-methylbenzylamine, N-methyl-2-methylbenzylamine, N-methyl-2-(1′-naphthyl)-ethylamine, 2-methyl-2′-nitrobenzylamine hydrochloride, 2-methyl-4′-nitrobenzylamine hydrochloride, 1-methyl-3-phenylpropylamine, 2-(1′-naphthyl)ethylamine, 2,(2′-naphthyl)ethylamine, phenylalaminol, (R)1-phenyl-3-aminobutane, 2-phenylglycinol, 1-phenylpropylamine, 2-phenyl-1-propylamine, (S)-prolinol, 1-threoninol, N-acetyl-2-phenylglycinol, dinaphthylprolinol, 2-methylpiperazine, piperidinol, quinuclidinol and combinations thereof;

23. The method of claim 7, wherein the amount of the racemate is at least 0.0001 mmol greater than the amount of said resolving agent.

24. A method of crystallization of diastereomeric salts, said method comprising the steps of:
(a) identifying at least one of a plurality of resolving agents and at least one of a plurality of solvents for separating enantiomeric isomers from a racemate, wherein the identification of the at least one of a plurality of resolving agents and the at least one of a plurality of solvents comprises the steps of:
(i) adding a predetermined quantity of the at least one resolving agent and a predetermined quantity of the at least one solvent to each of a plurality of containers arranged in an array, wherein each of the plurality of containers comprises a unique combination of the at least one resolving agent and the at least one solvent,
(ii) adding to each of the plurality of containers a predetermined quantity of the racemate to form a first combination of the at least one resolving agent, the at least one solvent and the racemate,
(iii) heating the first combinations in each of the plurality of containers to a predetermined temperature, wherein said predetermined first temperature is less than 80° C.,
(iv) cooling the heated first combinations in each of the plurality of containers to a predetermined second temperature, wherein said predetermined second temperature is greater than −4° C.,
(v) determining whether diastereomeric crystals are formed in each of the plurality of containers,
(vi) selecting at least one of the plurality of containers comprising the diastereomeric crystals,
(vii) optionally separating the diastereoisometric crystals from the first combination in each of the plurality of selected containers,
(viii) optionally isolating an isomer from the separated diastereomeric crystals in each of the plurality of selected containers,
(ix) evaluating the purity of the diastereoisometric crystals or of the isomer in each of the plurality of containers,
(x) identifying the at least one of the plurality of containers corresponding to the isomer having a highest purity, and
(xi) selecting the first combination associated with the identified plurality of containers corresponding to the isomer having the highest purity;
(b) purifying the isolated isomers having the highest purity, wherein the purification comprises the steps of:
(i) identifying stoichiometric ratio of the at least one resolving agent, the at least one solvent and the racemate from the selected first combination corresponding to the isomer having the highest purity and a predetermined yield,
(ii) preparing a plurality of third combinations comprising a predetermined amount of the at least one resolving agent, the at least one solvent and the racemate having the identified stoichiometric ratio, wherein each of the plurality of third combinations is prepared in the plurality of second collection containers,
(iii) heating the plurality of third combinations in each of the plurality of second collection containers to the predetermined temperature,
(iv) stirring the heated plurality of third combinations in each of the plurality of second collection containers to form a homogenous mixture,
(v) cooling the plurality of third combinations in each of the plurality of second collection containers to a temperate equal to or greater than −4° C.,
(vi) initiating formation of crystals of enantiomeric salt in each of the plurality of second collection containers,
(vii) filtering the plurality of third combinations in each of the plurality of second collection containers to separate the crystals of enantiomeric salt from a filtrate, wherein the filtrate comprises the at least one resolving agent, the at least one solvent and the racemate, wherein the filtrate from in each of the plurality of second collection containers is collected in a plurality of third collection containers,
(viii) measuring the purity of the separated crystals of the enantiomeric salt from each of the plurality of second collection containers,
(ix) comparing the measured purity of the separated crystals of the enantiomeric salt from each of the plurality of second collection containers to the predetermined purity,
(x) when the measured purity in (c)(viii) is less than the predetermined purity, adding the separated crystals from step (c)(vii) to the plurality of second collection containers,
(xi) heating a predetermined quantity of the at least one solvent to about its boiling point,
(xii) adding the heated solvent from step (c)(xi) to the plurality of second collection containers,
(xiii) stirring the contents in each of the plurality of second collection containers,
(xiv) cooling the contents in each of the plurality of second collection containers to room temperature,
(xv) initiating formation of crystals of enantiomeric salt in each of the plurality of second collection containers,
(xvi) collecting the crystals of enantiomeric salt in each of the plurality of second collection containers, and
(xvii) repeating steps (c)(vii)-(c)(xvi) until the measured purity of the separated crystals of the enantiomeric salt in at least one of the plurality of second collection containers is substantially the same as the predetermined purity; and
(c) optimizing the stoichiometric ratio of the racemate to the at least one resolving agent and concentration of the at least one solvent, wherein the optimizing step comprises:
(i) sorting the evaluated purity of the isomer from step (a)(ix) in an ascending or a descending order of purity,
(ii) selecting at least three of the isomers associated with the sorted purity, wherein the purity of the selected isomers rank at least first three in the sorted purity,
(iii) identifying the at least one resolving agent, the at least one solvent and the racemate associated with each of the selected isomers,
(iv) identifying a plurality of stoichiometric ratios of the at least one resolving agent, the at least one solvent and the racemate from the first combination corresponding to each of the selected isomers,
(v) preparing a plurality of fourth combinations, wherein each of the plurality of fourth combinations comprises a predetermined amount of the at least one resolving agent, the at least one solvent and the racemate having at least one of the plurality of identified stoichiometric ratios, wherein each of the plurality of fourth combinations is prepared in a plurality of fourth collection containers,
(vi) heating each of the plurality of fourth combinations in each of the plurality of fourth collection containers to the predetermined temperature,
(vii) stirring the heated plurality of fourth combinations in each of the plurality of fourth collection containers to form a homogenous mixture,
(viii) cooling each of the plurality of fourth combinations in each of the plurality of fourth collection containers to room temperature,
(ix) initiating formation of crystals of enantiomeric salt in each of the plurality of fourth collection containers,
(x) filtering each of the plurality of fourth combinations to separate the crystals of enantiomeric salt from a filtrate, wherein the filtrate comprises the at least one resolving agent, the at least one solvent and the racemate, wherein the filtrate from each of the plurality of fourth combinations is collected in a plurality of fifth collection containers,
(xi) measuring the purity of the separated crystals of the enantiomeric salt from each of the plurality of fourth combinations,
(xii) comparing the measured purity of the separated crystals of the enantiomeric salt from each of the plurality of fourth combinations to a predetermined purity,
(xiii) when the measured purity in (c)(xi) is less than the predetermined purity value, adding the separated crystals of the enantiomeric salt from each of the plurality of fourth combinations to the corresponding fourth collection container,
(xiv) heating a predetermined quantity of each at least one solvent identified in (c)(iii) for each of the selected isomers to about its boiling point,
(xv) adding the heated solvent(s) from step (c)(xiv) to each of the plurality of fourth collection containers,
(xvi) stirring the contents in each of the plurality of fourth collection containers,
(xvii) cooling the contents in each of the plurality of fourth collection containers to room temperature,
(xviii) initiating formation of crystals of enantiomeric salt in each of the plurality of fourth collection containers,
(xix) collecting the crystals of enantiomeric salt from each of the plurality of third collection containers,
(xx) for each isomer, repeating steps (c)(x)-(c)(xix) until the measured purity of the separated crystals of the enantiomeric salt from each of the plurality of third collection containers corresponds to a predetermined purity,
(xxi) identifying the container(s) within the plurality of containers corresponding to the crystals having a lowest crystallization time, highest yield and highest purity, and
(xxii) identifying a preferred combination from the plurality of fourth combinations, wherein the preferred combination is the stoichiometric ratio and the identity of the at least one resolving agent, the at least one solvent and the racemate within the container(s) requiring the least number of repetitions of step (c)(xx), and having the lowest crystallization time, highest yield and highest purity.

25. The method of claim 24, wherein said at least one solvent is selected from the group consisting of 90 percent acetone, methyl ethyl ketone (2-butanone), i-butanol, 2-propanol, 90 percent 2-propanol, methanol, 80 percent methanol, ethanol, 96 percent ethanol, water, 1-propanol, 85 percent 1-propanol, acetonitrile, ethyl acetate, dichloromethane, chloroform, p-dioxane, methyl-t-butyl ether, toluene and tetrahydrofuran.

26. The method of claim 24, wherein said at least one resolving agent, is at least one of a chirally pure acid or a chirally pure base and is selected from the group consisting of tartaric acid, pyroglutamic acid, di-p-tolulo-tartaric acid, mandelic acid, malic acid, camphorsulphonic acid, dibenzoyl-tartaric acid, deoxycholic acid (+), camphoric acid (+), quinic acid (−), aspartic acid (+), glutamic acid, 1,3,4,6-diisopropylidine-2-ketogluconic acid (−), acetylmandelic acid, N-acetyl-1-hydroxyproline, N-acetyl-1-leucine, acetyl-3-mercapto-2-methylpropionic acid, 3-acetylmercapto-2-methylpropionyl-1-proline, N-acetyl-D-3-(2-naphthyl)-alanine, (R)-acetylthio-2-methylpropionyl chloride, N-acetyl-1-phenylalanine, N-acetyl-1-tyrosinamide, D-alanine, 1-aminoadipic acid, (R)-2-aminobutyric acid, (1R,4S)-4-aminocyclopent-2-ene-1-carboxylic acid, (1S,4R)-4-aminocyclopent-2-ene-1-carboxylic acid, S-2-amino-3,3-dimethylbutyric acid, 1-tert-leucine, 1,2-amino-2-methyl-3-(3′,4′-dimethoxyphenyl)-propionitrile HCl, 1-2-amino-2-methyl-3-(3′,4′-dihydroxyphenyl)-propionic acid, (R)-2-amino-4-phenylbutane, D-arginine, D-aspartic acid, D-2-azidophenylacetic acid, D-2-azidophenylacetyl chloride, (1S,2R)-cis-2-benzamido-cyclohexane-carboxylic acid, (1R,2S)-cis-2-benzamido-cyclohexane-carboxylic acid, benzyl-(R) & (S)-mandelate, benzyl-2-tosyloxypropionate, N-2-BOC-D-alanine, N-2-BOC-1-aminoadipic acid, 3-(R)-BOC-aminocyclopent-4-ene-1-(S)-carboxylic acid, 3-(S)-BOC-aminocyclopent-4-ene-1-(R)-carboxylic acid, N-2-BOC-D-arginine hydrochloride, N-2-BOC-D-aspartic acid, N-2-BOC-3-(4-biphenyl)alanine, N-2-BOC-N-6-CBZ-D-lysine, N-2-BOC-3-(4-chlorophenyl)-alanine, N-2-BOC-cyclohexylalanine, N-2-BOC-1-cyclohexylalanine methyl ester, N-2-BOC-3,3-diphenylalanine, N-2-BOC-3-(4-fluorophenyl)-alanine, N-2-BOC-D-glutamic acid 1-benzyl ester, N-2-BOC-D-histidine, N-2-BOC-3-(4-iodophenyl)-alanine, N-3-BOC-D-leucine, N-3-BOC-1-tert-leucine DCHA salt, (1S)-camphanic acid, (1R)-camphorsulfonic acid, (1S)-camphorsulfonic acid, 2-methylbenzylamine, N-2-BOC-D-methionine, N-2-BOC-3-(1′-naphtyl)alanine, N-2-BOC-3-(2′-naphtyl)alanine, N-2-BOC-3-(4′-nitrophenyl)alanine, N-2-BOC-1-octahydroindole-2-carboxylic acid, N-2-BOC-3-(pentafluorophenyl)-alanine, N-2-BOC-D-phenylalanine, N-BOC-D-proline, N-1-BOC-D-3-(2′-pyridyl)alanine, N-2-BOC-1-3-(2′-pyridyl)alanine, N-2-BOC-D-3-(3-pyridyl)alanine, N-1-BOC-1-3-(3′-pyridyl)alanine, N-2-BOC-D-serine, N-BOC-1,2,3,4-tetrahydroisoquinoline-(3R)-carboxylic acid, N-BOC-1,2,3,4-tetrahydroisoquinoline-(3S)-carboxylic acid, N-2-BOC-3-(4′-thiazolyl)alanine, N-BOC-D-threonine, N-2-BOC-N-8-tosyl-D-arginine, N-2-BOC-D-tryptophan, N-2-BOC-D-tyrosine, N-2-BOC-D-tyrosine methyl ester, N-2-BOC-D-valine, 2-bromobutyric acid, 2-bromohexadecanoic acid, (R)-2-bromo-2-phenylacetic acid, 2-bromopropionic acid, butyl-(S)-2-chloropropionate, (2R,3S)-butyl-2,3-epoxybutyrate, (R)-butyl-2,3-epoxybutyrate, (S)-tert-butyl-3-hydroxybutyrate, (S)-butyl-lactate, N-butyl-(R)-2-methyl-2-hydrazino-3-(3′-methoxy-4′-hydroxyphenyl)-propionate, N-CBZ-D-alanine, N-CBZ-D-arginine, N-CBZ-D-aspartic acid, N-CBZ-O-tert-butyl-D-serine, CBZ-1-cyclohexylalanine, N-CBZ-D-glutamic acid, N-CBZ-D-histidine, N-CBZ-D-leucine, N-CBZ-1-tert-leucine DCHA salt, N-CBZ-D-methionine, N-2-CBZ-D-3-(2′-naphthyl)alanine, N-2-CBZ-ornithine, N-2-CBZ-D-phenylalanine, N-2-CBZ-D-proline, N-2-CBZ-D-serine, N-2-CBZ-D-threonine, N-2-CBZ-D-tryptophan, N-2-CBZ-D-tyrosine, N-2-CBZ-D-valine, (R)-2-chlorobutyric acid, 3-chloromandelic acid, 4-chloromandelic acid, 1-((S)-3-chloro-2-methylpropionyl)-1-proline, (R)-2-(4′-chlorophenoxy)-propionic acid, 3-(4′-chlorophenyl)alanine, 2-(4′-chlorophenyl)-3-phenylpropionic acid, chlorophos, 2-chloropropionic acid, (S)-2-chloropropionic acid sodium salt (50 percent solution), cyclohexylalanine, cyclohexylglycine, cyclophos, D-cysteine, D-cysteine hydrochloride monohydrate, D-cysteine, dibenzoyl-tartaric acid, 1-3-(3′,4′-dichlorophenyl)-alanine, diethyl-1-tartrate, D-1-dihydrophenylglycine, D-1-dihydrophenylglycine chloride hydrochloride, D-(3′,4′-dihydroxy)-1-phenylglycine, diisopropyl-tartrate, dimethyl-tartrate, 2-3-diphenylpropionic acid, di-p-toluoyl-tartaric acid, ethyl-(R)-2-(N-acetylamino)-2,4-dimethylpentanoate, ethyl-(R)-2-(N-acetylamino)-2-methyl-3-phenylpropionate, ethyl-4-bromo-3-hydroxybutyrate, ethyl-4-chloro-3-hydroxybutyrate, ethyl-(S)-2-chloropropionate, ethyl-2-3-dihydroxybutyrate, ethyl-2-3-dihydroxy-3-phenylpropionate, (R)-ethyl-3-hydroxybutyrate, ethyl-2-hydroxy-2-phenylacetate, ethyl-(R)-2-hydroxy-4-phenybutyrate, ethyl-3-hydroxy-3-phenylpropionate, (R)-ethyl-4-iodo-3-hydroxybutyrate, N-(1-phenylethyl)-phtalimide, D-phenylglycine, N,N,N′,N′-tetramethyl-tartaric acid, thiazolidine-4-carboxylic acid, 3-(2-thienyl)-alanine, D-allo-threonine, valine, N-methylglucamine (−), α-methylbenzylamine, cinochonidine (−), ephedrine (−), hydroquinidine (+), N-benzyl-α-methylbenzylamine, brucine (−), strychnine (−), pseudoephedrine (+), qunidine, quinine (−), cinchonine (+), threo 2-amino-1-(p-nitrophenyl)-1,3-propanediol, 2-amino-1-butanol, methylephedrine (−), α-1-naphthylethyl amine, dehydroabietyl amine, 2-amino-1-phenyl-1,3-propanediol, D-alaninamide, 2-amino-1-propanol, 2-aminobutanol, erythro-2-amino-1,2-diphenylethanol, (S)-1-aminoindane, cis-(1S,2R)aminoindan-2-ol, 1-amino-2-(methoxymethyl)-pyrrolidine, 2-amino-3-methyl-1-butanol, 2-amino-3-methyl-1-pentanol-isoleucinol, 2-amino-4-methyl-1-pentanol-leucinol, 2-amino-1-[4′-(methylthio)-phenyl]-1,3-propanediol, 2-amino-1-phenylethanol, 1-amino-2-propanol, 1-aminotetralin and N-propyl derivative, 2-aminotetralin and N-propyl derivative, N-benzyl-3-aminopyrrolidine, benzyl-benzyl amine, benzyl-4-chlorobenzylamine, cis-N-benzyl-2-(hydroxymethyl)cyclohexylamine, N-benzyl-3-hydroxypyrrolidine, N-benzyl-2-methylbenzylamine, N-benzylamine-methylbenzylamine hydrochloride, 2-benzyl-2-methylbenzylamine, 2-benzyl-3′-methylbenzylamine, 2-benzyl-4′-methylbenzylamine, N-benzyl-1-(1′-naphthyl)ethylamine hydrochloride, bis(methoxymethyl)pyrrolidine, bis{1-[1-naphthyl]ethyl}amine hydrochloride, bis(1-phenylethyl)amine hydrochloride, N,N-bis-[1-phenylethyl]phthalamic acid, N-2-BOC-cyclohexylglycine, BOC-isoleucinol, BOC-phenylalaminol, BOC-prolinol, N-butyl-2-amino-2-methyl-3-(3′,4′-dihydroxyphenyl)-propionate, CBZ-1-cyclohexylalaminol, N-2-CBZ-D-3-(1′naphthyl)alaminol, N-2-CBZ-D-3-(2′naphthyl)alaminol, N-1-phenylalaminol, 2-(2′-chlorobenzyl)benzyl-amine, 2-(3′-chlorobenzyl)benzyl-amine, 2-(4′-chlorobenzyl)benzylamine, (S)-cyclohexylalaminol, 1,2-diaminocyclohexane, (S)-2,6-diamino-1-hexanol (1-lysinol), 1,2-diaminopropane, 2,2-dibenzyl-2-hydroxy-1-methylethylamine, N,N-dibenzylphenylalaminol, N-(3,4-dimethoxybenzyl)-1-phenylethylamine, 3,3-dimethyl-2-aminobutane, N,N-dimethyl-1-methylbenzylamine, N,N-dimethyl-2-(1′-naphthyl)ethylamine, N-(3′,4′-dinitrobenzoyl)-2-methylbenzylamine, N-(3′,5′-dibenzoyl)-1-(1-naphthyl)ethylamine, 1,2-diphenyl-1,2-ethanediamine, 2,2-diphenyl-2-hydroxy-1-methylethylamine, N,N′-ditosyl-1,2-diphenyl-1,2-ethanediamine, 2,2-diphenyl-2-hydroxy-1-methylethylamine, N,N′-ditosyl-1,2′-diphenyl-1,2-ethanediamine, diphenylvalinol, diphenylprolinol, ethyl-(R)-2-amino-2-methyl-3(3′,4′-dimethoxyphenyl)propionate, ethyl (R)-2-amino-2-methyl-3-phenylpropionate, 3-hydroxypyrrolidine, 3-hydroxypyrrolidine HCl, isopropyl-2-methylbenzylamine, 1-tert-leucinol, 1-tert-leucinol hydrochloride, 1-methioniol, 5-methoxy-2-aminotetralin, N-propyl-5-methoxy-2-aminotetralin, 6-methoxy-2-aminotetralin and N-propyl-6-methoxy-2-aminotetralin, 7-methoxy-2-aminotetralin and N-propyl, 8-methoxy-2-aminotetralin and N-propyl, (S)-2-(methoxymethyl)pyrrolidine, (S)-2-(methylamino)propiophenone, D-N-methylamphetamine, 2-(4′-methylbenzyl)benzylamine, 2-(4′methylbenzyl)-N′N′dimethylbenzylamine, 2-(4′-methylbenzyl)-N-hydroxyethyl-benzylamine, 2-methyl-3′-bromobenzylamine, 2-methyl-4′-bromobenzylamine, 2-methyl-4′-bromobenzylamine hydrochloride, 2-methyl-4′-chlorobenzylamine, 2-methyl-2′-methoxybenzylamine, 2-methyl-3′-methoxybenzylamine, 1-methyl-3′-methoxybenzylamine, 2-methyl-4′-methoxybenzylamine, 2-methyl-4′-methylbenzylamine, N-methyl-2-methylbenzylamine, N-methyl-2-(1′-naphthyl)-ethylamine, 2-methyl-2′-nitrobenzylamine hydrochloride, 2-methyl-4′-nitrobenzylamine hydrochloride, 1-methyl-3-phenylpropylamine, 2-(1′-naphthyl)ethylamine, 2,(2′-naphthyl)ethylamine, phenylalaminol, (R)1-phenyl-3-aminobutane, 2-phenylglycinol, 1-phenylpropylamine, 2-phenyl-1-propylamine, (S)-prolinol, 1-threoninol, N-acetyl-2-phenylglycinol, dinaphthylprolinol, 2-methylpiperazine, piperidinol, quinuclidinol and combinations thereof.

27. The method of claim 24, wherein the resolving agent is Tartaric acid

28. The method of claim 24, wherein the solvent is ethanol or 96 percent ethanol.

29. The method of claim 24, wherein about 0.03 mmol of the racemate is added to each container.
